(19) United States
(12) Patent Application Publication
(10) Pub. No.: US 2024/0117384 A1
(43) Pub. Date: Apr. 11, 2024

(54) METHOD FOR CONVERTING NUCLEIC ACID SEQUENCE OF CELL SPECIFICALLY CONVERTING NUCLEIC ACID BASE OF TARGETED DNA USING CELL ENDOGENOUS DNA MODIFYING ENZYME, AND MOLECULAR COMPLEX USED THEREIN

(71) Applicant: NATIONAL UNIVERSITY CORPORATION KOBE UNIVERSITY, Kobe (JP)

(72) Inventors: Keiji NISHIDA, Kobe (JP); Akihiko KONDO, Kobe (JP); Takayuki ARAZOE, Kobe (JP); Shin YOSHIOKA, Kobe (JP)

(73) Assignee: NATIONAL UNIVERSITY CORPORATION KOBE UNIVERSITY, Kobe (JP)

(21) Appl. No.: 18/539,833

(22) Filed: Dec. 14, 2023

Related U.S. Application Data
(62) Division of application No. 16/496,311, filed on Sep. 20, 2019, now Pat. No. 11,845,953.

(30) Foreign Application Priority Data
Mar. 22, 2017 (JP) 2017-056727

(51) Int. Cl. C12N 15/90; C12N 9/22; C12N 15/11; C12N 15/85

(52) U.S. Cl. CPC C12N 15/907; C12N 9/22; C12N 15/11; C12N 15/85; C12N 2310/20; C12N 2800/80

(57) ABSTRACT

Provided is a method for altering a targeted site of a DNA in a cell, including a step of stimulating the cell with a factor inducing a DNA modifying enzyme endogenous to the cell, and bringing a complex of a nucleic acid sequence-recognizing module specifically binding to a target nucleotide sequence in a given DNA and a DNA modifying enzyme-binding module bonded to each other into contact with the DNA to convert one or more nucleotides in the targeted site to other one or more nucleotides or delete one or more nucleotides, or insert one or more nucleotides into the targeted site.

### CROSS-REFERENCE TO RELATED APPLICATIONS

This patent application is a divisional of copending U.S. patent application Ser. No. 16/496,311, filed on Sep. 20, 2019, which is the U.S. national phase of International Patent Application No. PCT/JP2018/011198, filed Mar. 20, 2018, which claims the benefit of Japanese Patent Application No. 2017-056727, filed Mar. 22, 2017, which are incorporated by reference in their entireties herein.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: a 159,359 byte Extensible Markup Language (xml) file named “769742 ST26 Sequence Listing,” created Dec. 14, 2023.

### TECHNICAL FIELD

The present invention relates to a method for altering a nucleic acid sequence, which enables alteration of a nucleic acid base in a particular region of intracellular target DNA, without introducing an exogenous DNA modifying enzyme or a nucleic acid encoding same into the cell, and a complex of a nucleic acid sequence-recognizing module and a DNA modifying enzyme-binding module to be used therefor.

### BACKGROUND ART

In recent years, genome editing is attracting attention as a technique for altering the object gene and genome region in various species. Conventionally, as a method of genome editing, a method utilizing an artificial nuclease comprising a molecule having a sequence-independent DNA cleavage ability and a molecule having a sequence recognition ability in combination has been proposed (non-patent document 1).

For example, a method of performing recombination at a target gene locus in DNA in a plant cell or insect cell as a host, by using a zinc finger nuclease (ZFN) wherein a zinc finger DNA binding domain and a non-specific DNA cleavage domain are linked (patent document 1), a method of cleaving or modifying a target gene in a particular nucleotide sequence or a site adjacent thereto by using TALEN wherein a transcription activator-like (TAL) effector which is a DNA binding module that the plant pathogenic bacteria Xanthomonas has, and a DNA endonuclease are linked (patent document 2), a method utilizing CRISPR-Cas9 system wherein DNA sequence CRISPR (Clustered Regularly interspaced short palindromic repeats) that functions in an acquired immune system possessed by eubacterium and archaebacterium, and nuclease Cas (CRISPR-associated) protein family having an important function along with CRISPR are combined (patent document 3) and the like have been reported. Recently, Cpf1 was reported as a new endonuclease for a CRISPR-Cas system (non-patent document 2). Furthermore, a method of cleaving a target gene in the vicinity of a particular sequence, by using artificial nuclease wherein a PPR protein constituted to recognize a particular nucleotide sequence by a continuation of PPR motifs each consisting of 35 amino acids and recognizing one nucleic acid base, and nuclease are linked (patent document 4) has also been reported.

Recently, moreover, the present inventors reported that a genome sequence was successfully altered, without DSB, by nucleic acid base conversion in a region containing a particular DNA sequence in various biological species including yeast and Escherichia coli, by using deaminase that catalyzes a deamination reaction and by introducing a complex of the deaminase linked to a molecule having a DNA sequence recognition ability into the host cell (patent document 5, non-patent document 3).

[Document List]

[Patent documents]
patent document 1: JP-B-4968498
patent document 2: National Publication of International Patent Application No. 2013-513389
patent document 3: National Publication of International Patent Application No. 2010-519929
patent document 4: JP-A-2013-128413
patent document 5: WO 2015/133554

### Non-Patent Document

non-patent document 1: Kelvin M Esvelt, Harris H Wang (2013) Genome-scale engineering for systems and synthetic biology, Molecular Systems Biology 9: 641 non-patent document 2: Bernd Zetsche et al. (2015) Cpf1 Is a Single RNA-Guided Endonuclease of a Class 2 CRISPR-Cas System,

Cell 163: 759-771 non-patent document 3: Nishida Keiji et al. (2016) Targeted nucleotide editing using hybrid prokaryotic and vertebrate adaptive immune systems, Science 6: 353(6305)

### SUMMARY OF THE INVENTION

### Problems to be Solved by the Invention

While the above-mentioned genome editing techniques proposed so far presuppose introduction of an exogenous DNA modifying enzyme into the cell, they are associated with problems of side effects such as cytotoxicity and the like and delivery of the DNA modifying enzyme into the cell or target DNA site, which are caused by the use of the DNA modifying enzyme. It is therefore an object of the present invention to provide a method of novel DNA editing, particularly, genome editing, capable of increasing safety by utilizing a cell-endogenous DNA modifying enzyme and avoiding restriction of delivery, and a complex therefor of a nucleic acid sequence-recognizing module and a DNA modifying enzyme-binding module.

### Means of Solving the Problems

The present inventors produced a complex in which a nucleic acid sequence-recognizing module targeting the object DNA sequence is imparted with a function to bind to a cell-endogenous DNA modifying enzyme, introduced the complex into the cell, and cultured the cell in the presence of a factor inducing the DNA modifying enzyme. As a result, they successfully introduced a mutation into the target nucleotide sequence of the object gene and the vicinity thereof without using an exogenous DNA modifying enzyme.

The present inventors have conducted further studies based on these findings and completed the present invention.

Therefore, the present invention is as described below.
[1] A method for altering a targeted site of a DNA in a cell, comprising a step of stimulating the cell with a factor inducing a DNA modifying enzyme endogenous to the cell, and bringing a complex of a nucleic acid sequence-recognizing module specifically binding to a target nucleotide sequence in a given DNA and a DNA modifying enzyme-binding module bonded to each other into contact with the DNA to convert one or more nucleotides in the targeted site to other one or more nucleotides or delete one or more nucleotides, or insert one or more nucleotides into said targeted site.
[2] The method of [1], wherein the aforementioned targeted site is altered without cleaving at least one of the strands of the aforementioned DNA.
[3] The method of [1] or [2], wherein the aforementioned nucleic acid sequence-recognizing module is selected from the group consisting of a CRISPR-Cas system wherein at least one DNA cleavage ability of Cas is inactivated, a zinc finger motif, a TAL effector and a PPR motif.
[4] The method of any of [1] to [3], wherein the aforementioned nucleic acid sequence-recognizing module is a CRISPR-Cas system wherein at least one DNA cleavage ability of Cas is inactivated.
[5] The method of any of [1] to [4], wherein the aforementioned DNA modifying enzyme-binding module is selected from the group consisting of an antibody against the DNA modifying enzyme, a peptide aptamer against the DNA modifying enzyme and a nucleic acid aptamer against the DNA modifying enzyme.
[6] The method of any of [1] to [4], wherein the aforementioned DNA modifying enzyme-binding module is at least one kind selected from the group consisting of Vif, Bet protein, TopoIIβ, IQGAP2 and ZNF335 and fragments thereof.
[7] The method of any of [1] to [6], wherein a target enzyme of the aforementioned DNA modifying enzyme-binding module is deaminase.
[8] The method of [7], wherein the aforementioned deaminase is a protein belonging to the APOBEC family.
[9] The method of [7] or [8], wherein the complex of the nucleic acid sequence-recognizing module bonded to the DNA modifying enzyme-binding module further comprises a base excision repair inhibitor bonded thereto.
[10] The method of any of [1] to [9], wherein the aforementioned factor inducing the DNA modifying enzyme includes one or more selected from the group consisting of interferon, an inhibitor of succinic acid dehydrogenase and hypoxic condition.
[11] The method of any of [1] to [10], wherein the aforementioned DNA and the aforementioned complex are contacted by introducing a nucleic acid encoding the complex into the aforementioned cell and culturing the cell to cause expression of the complex in the cell.
[12] The method of any of [1] to [11], wherein the cell is stimulated by the factor inducing the DNA modifying enzyme by incubating the cell in the presence of the factor.
[13] The method of any of [1] to [12], wherein the aforementioned cell is a vertebrate cell.
[14] The method of [13], wherein the aforementioned vertebrata cell is a mammalian cell.
[15] The method of any of [1] to [14], wherein the aforementioned DNA is a double stranded DNA.
[16] A complex of a nucleic acid sequence-recognizing module specifically binding to a target nucleotide sequence in a DNA and a DNA modifying enzyme-binding module bonded to each other, wherein the nucleic acid sequence-recognizing module is a CRISPR-Cas system wherein at least one DNA cleavage ability of Cas is inactivated, wherein the complex converts one or more nucleotides in the targeted site to other one or more nucleotides or deletes one or more nucleotides, or inserts one or more nucleotides into said targeted site.
[17] A nucleic acid encoding the complex of [16].
[18] An agent for altering a targeted site of a DNA comprising the complex of or the nucleic acid of [17].
[19] A method for altering a targeted site of a double stranded DNA in a cell, comprising a step of stimulating the cell with a factor inducing a DNA modifying enzyme endogenous to the cell, and bringing a nucleic acid sequence-recognizing module specifically binding to a target nucleotide sequence in a given double stranded DNA into contact with the double stranded DNA to convert one or more nucleotides in the targeted site to other one or more nucleotides or delete one or more nucleotides, or insert one or more nucleotides into said targeted site.

### Effect of the Invention

According to the DNA editing of the present invention, the risk of side effects is reduced since an exogenous factor is not used in the DNA modification reaction. In addition, delivery efficiency can be improved since the construct used for DNA editing can be miniaturized. Utilizing a cell-endogenous DNA modifying enzyme, moreover, the activity can be controlled by a transient action and the risk of off-target action can be reduced.

### BRIEF DESCRIPTION OF THE DRAWINGS

FIG. 1 is a schematic showing of the mechanism of the alteration method of the targeted site of DNA used in the Examples of the present invention. In FIG. 1, IFN is interferon (factor inducing, as antivirus factor, particular defense gene expression), IFN-inducible endogenous deaminase is an antiviral deaminase group (Apobec etc.) showing IFN-induced expression, and dVif (Vif variant) is an adapter protein bonded to endogenous deaminase.

FIG. 2 is a schematic showing of the plasmid for DNA editing used in the Examples.

FIG. 3 is a schematic showing of the plasmid for DNA editing used in the Examples.

### DESCRIPTION OF EMBODIMENTS

The present invention provides a method for altering a targeted site in the DNA in a cell by utilizing a DNA modifying enzyme endogenous to the cell (to be also referred to as “cell-endogenous” in the present specification) to convert the target nucleotide sequence and nucleotides in the vicinity thereof in the DNA in the cell to other nucleotides (hereinafter to be also referred to as “the method of the present invention”). As used herein, “endogenous to cell”, “cell-endogenous” mean native presence in the cell.

The method of the present invention is characterized by a step in which the cell is stimulated with a factor inducing a cell-endogenous DNA modifying enzyme (hereinafter to be also referred to as “DNA modifying enzyme inducer”) and a complex in which a nucleic acid sequence-recognizing module that specifically binds to the target nucleotide sequence in the DNA and a DNA modifying enzyme-binding module are bonded to each other (hereinafter to be also referred to as “the complex of the present invention”) is contacted with the DNA in the cell to convert the targeted site, i.e., the target nucleotide sequence and nucleotides in the vicinity thereof, to other nucleotides.

In the present invention, the “alteration” of a DNA means that a nucleotide (e.g., dC) on a DNA strand is converted to other nucleotide (e.g., dT, dA, dG or dU), or deleted, or a nucleotide or a nucleotide sequence is inserted between certain nucleotides on a DNA strand. The DNA to be altered is not particularly limited as long as it is a DNA that the cell has (or present in the cell). It may be a cell-endogenous DNA (e.g., chromosome DNA, mitochondria DNA, chloroplast DNA; hereinafter these are to be comprehensively referred to as “genomic DNA”) or an exogenous DNA (e.g., DNA derived from virus infected with cell). The aforementioned DNA may be a single strand DNA or a double stranded DNA, preferably a double stranded DNA. As the double stranded DNA, preferred is genomic DNA. The “targeted site” of a DNA means the whole or partial “target nucleotide sequence”, which a nucleic acid sequence-recognizing module specifically recognizes and binds to, or the vicinity of the target nucleotide sequence (one or both of 5′ upstream and 3′ downstream). The “target nucleotide sequence” means a sequence to which a nucleic acid sequence-recognizing module in the DNA binds.

In the present invention, the “DNA modifying enzyme” means a cell-endogenous enzyme capable of modifying DNA, and the modification directly or indirectly causes alteration of DNA. Examples of such DNA modification reaction include a reaction to cleave single strand or double strand of DNA (hereinafter to be also referred to as “DNA strand cleavage reaction”), a reaction to convert a substituent on the purine or pyrimidine ring of a nucleic acid base to other group or atom, which is a reaction not directly involving cleavage of DNA strand (hereinafter to be also referred to as “nucleic acid base conversion reaction”) (e.g., deamination reaction of base), a reaction to hydrolyze N-glycoside linkage of DNA (hereinafter to be also referred to as “base excision reaction”) and the like.

In the present invention, the “DNA modifying enzyme inducer” means a molecule that can directly or indirectly increase the expression of cell-endogenous DNA modifying enzyme and/or a factor that can activate the DNA modifying enzyme (including molecule, physicochemical stimulation such as oxygen concentration, light, UV, temperature, acid, alkali and the like, and the like). The DNA modifying enzyme inducer to be used in the method of the present invention is not particularly limited as long as it has such function. Examples thereof include protein (including peptide, hereinafter the same) (e.g., transcription factor, interferon (IFN), interleukin, Mitogen etc.), low-molecular-weight compound and the like. The DNA modifying enzyme inducer used may be commercially available or one produced by a well known method.

Interferon (IFN) is a protein secreted by cells in response to the invasion of foreign substances such as pathogen (particularly virus), tumor cell and the like, and stimulation of cells with IFN induces expression of antiviral proteins (e.g., proteins belonging to APOBEC (apolipoprotein B mRNA-editing enzyme catalytic polypeptide-like) family and the like). The interferon to be used in the present invention is not particularly limited and TYPE I interferon (e.g., IFN-α, IFN-β, IFN-ω, IFN-ε, IFN-κ), TYPE II interferon (e.g., IFN-γ), TYPE III interferon (e.g., IFN-λ) and the like can be mentioned. Particularly, TYPE I interferon is preferable, and IFN-α and IFN-β are preferable. Interferon may be a natural type or a gene recombinant type, or a pegylated interferon in which a macromolecular form such as polyethylene glycol (PEG) or the like is bonded. When interferon is used, the host cell and the organism from which the interferon is derived are preferably the same (e.g., when human cell is used, human interferon is preferably used). An IFN production-inducing factor may also be used. Examples of such factor include (quasi)infection with virus and the like, vaccine, exogenous DNA or RNA, double stranded RNA analogue [poly(I:C)] (e.g., Trapp Si, et al., (2009) J. Virol, 83(2):884-895), interferon gene stimulator, TANK-binding kinase 1 and the like.

Examples of the interleukin to be used in the present invention include IL-2, IL-7, IL-15, IL-27 and the like known to be able to induce proteins belonging to the APOBEC family (hereinafter to be abbreviated as “APOBEC”) (particularly, proteins belonging to the APOBEC3 family (hereinafter to be abbreviated as “APOBEC3”)), namely, to increase the expression and/or activity of the proteins.

Examples of the mitogen to be used in the present invention include phorbol ester (e.g., phorbol myristate acetate (PMA), phytohemagglutinin (PHA) etc.) known to be able to induce APOBEC (particularly, APOBEC3) (e.g., Stopak S. Kim, et al., (2007) J. Biol Chem., 282(6): 3539-3546; Rose KM1, et al., (2004) J. Biol Chem., 279(40):41744-41749) and the like.

Examples of the low-molecular-weight compound to be used in the present invention include the compounds described in JP-A-2011-231053, inhibitors of succinic acid dehydrogenase described in WO 2016-164889 (e.g., Atpenin A5, malonate, diazoxide (DZX), malate and oxaloacetate, 3-nitropropionic acid, nitroxyl, carboxin, TTFA etc.) and the like known to be able to induce APOBEC (particularly, APOBEC3).

The DNA modifying enzyme inducer is not limited to these and those of ordinary skill in the art can appropriately use known proteins and compounds, physicochemical stimulation and the like according to the kind of the target DNA modifying enzyme. Only one kind of the DNA modifying enzyme inducer may be used or two or more kinds thereof may be used (e.g., combined use of interferon and inhibitor of succinic acid dehydrogenase, combined use of interferon and hypoxic condition and the like).

The method for stimulating a cell with a DNA modifying enzyme inducer is not particularly limited. For example, a method including incubating the cell in the presence of a DNA modifying enzyme inducer can be mentioned. Specifically, it can be performed by adding a DNA modifying enzyme inducer to a medium or buffer for incubating the cells or, when the factor is a physicochemical stimulation such as hypoxia or the like, by incubating the cells under a condition with the presence of the stimulation. In addition, a method including introduction of a nucleic acid encoding a DNA modifying enzyme inducer (preferably DNA) into the cell and expression of the factor in the cell can be mentioned.

Also, the timing to start stimulation of the cell with a DNA modifying enzyme inducer is not particularly limited. For example, when the intracellular target DNA and the complex of the present invention are contacted by introducing a nucleic acid encoding the complex into the cell, it may be either before, after or simultaneously with the introduction step. In the method of the present invention, the period of DNA modification reaction can be adjusted by adjusting the period of stimulation of the cells with a DNA modifying enzyme inducer. Therefore, editing of the target sequence can be realized efficiently while avoiding the risk of off-target action in the host genome by stimulating the cells with the DNA modifying enzyme inducer for the period of time necessary for DNA modification reaction to occur and alteration of the targeted site to be fixed. From the aspect of easy adjustment of the period for cell stimulation, a method for incubating the cells in the presence of a DNA modifying enzyme inducer (for example, when the DNA modifying enzyme inducer is a protein, a low-molecular-weight compound or the like, a method for adding the factor to a medium or buffer) is preferable. The period for addition to the medium or buffer varies depending on the type of host cell, incubation conditions, the type of DNA modifying enzyme to be targeted, and the like. When the DNA to be modified is endogenous to the cell, about 2-3 days are considered to be necessary since at least several generations of cell division are generally necessary. On the other hand, when the DNA to be modified is exogenous DNA, the period can be shortened as compared with the intracellular DNA since cell division is not generally necessary. Those of ordinary skill in the art can appropriately determine a preferable expression induction period based on the culture conditions and the like to be used.

The content of the DNA modifying enzyme inducer to be added to the medium is not particularly limited as long as the target DNA is altered. When interferon is used as a DNA modifying enzyme inducer, it is added to the medium at preferably 10-100000IU (international unit), more preferably 100-20000IU, further preferably 500-5000IU. When Aptenin A5 is used as a DNA modifying enzyme inducer, it is added to the medium at preferably 0.5 μM-10 μM, more preferably 1 μM-3 μM. Those of ordinary skill in the art can appropriately determine a preferable content, titer, and the like based on the DNA modifying enzyme inducer to be used, cell type, culture conditions and the like.

When a DNA modifying enzyme inducer is a physicochemical stimulation, one preferable embodiment is a hypoxic condition. For example, it has been reported that proteins belonging to the APOBEC family can be activated when exposed to hypoxic conditions (e.g., WO 2016-164889). Examples of the method for exposing cells to hypoxic conditions include a method for incubating cells in a hypoxic state atmosphere and the like. Here, the “hypoxic state” means that the oxygen concentration is lower than the oxygen concentration in the atmosphere. Examples of such oxygen concentration include not more than 15%, preferably not more than 10%, more preferably not more than 5%, further preferably not more than 1%, and preferably not less than 0.1%.

Alternatively, when a nucleic acid (preferably DNA) encoding a DNA modifying enzyme inducer is introduced into a cell and the factor is expressed in the cell, it can be introduced into a cell in the same manner as a nucleic acid encoding the below-mentioned nucleic acid sequence-recognizing module and/or DNA modifying enzyme-binding module. When a DNA encoding a DNA modifying enzyme inducer is used, the DNA is placed under the control of an inducible regulatory region, substances capable of activating the regulatory region is added to and/or removed from the medium or buffer in which the cells are incubated to adjust the expression period of the DNA modifying enzyme inducer in the cell, whereby the period during which the DNA modification reaction occurs can be adjusted. As the “inducible regulatory region”, the regulatory region described later for regulation of the expression of the nucleic acid encoding the complex of the present invention can be used similarly.

In the present invention, the “nucleic acid sequence-recognizing module” means a molecule or molecule complex having an ability to specifically recognize and bind to a particular nucleotide sequence (i.e., target nucleotide sequence) on a DNA strand. Binding of the nucleic acid sequence-recognizing module to a target nucleotide sequence enables cell-endogenous DNA modifying enzyme to specifically act on a targeted site of a DNA via DNA modifying enzyme-binding module linked to said module.

In the present invention, the “DNA modifying enzyme-binding module” means a molecule or molecule complex having the ability to bind to a DNA modifying enzyme.

The complex of the present invention is a molecular complex containing a complex in which the above-mentioned nucleic acid sequence-recognizing module and DNA modifying enzyme-binding module are linked, and provided with specific nucleotide sequence recognition ability and a cell-endogenous DNA modifying enzyme. The “complex” here encompasses not only one constituted of multiple molecules, but also one having a nucleic acid sequence-recognizing module and DNA modifying enzyme-binding module in a single molecule, like a fusion protein.

In the present invention, the cell-endogenous DNA modifying enzyme as a binding target of a DNA modifying enzyme-binding module (hereinafter to be also referred to as “target enzyme”) is not particularly limited. Examples thereof include nuclease (e.g., endonuclease, exonuclease etc.), recombinase, DNA gyrase, DNA polymerase, DNA topoisomerase, telomerase, transposase, deaminase, DNA glycosylase and the like. From the viewpoint of reduced cytotoxicity, alteration of DNA is preferably performed not by a cleavage reaction of strand of double stranded DNA, but by a reaction that does not cleave at least one strand of double stranded DNA (e.g., nucleic acid base conversion reaction and base excision reaction on DNA). Examples of the DNA modifying enzyme that catalyzes nucleic acid base conversion reaction and base excision reaction include deaminase belonging to the nucleic acid/nucleotide deaminase superfamily that catalyzes a deamination reaction to convert an amino group to a carbonyl group, DNA glycosylase that catalyzes hydrolysis of N-glycoside linkage of DNA (e.g., thymine DNA glycosylase, oxoguanine glycosylase, alkyladenine DNA glycosylase (e.g., yeast 3-methyladenine-DNA glycosylase (MAGI)) and the like) and the like. Preferable examples of deaminase include cytidine deaminase capable of converting cytosine or 5-methylcytosine to uracil or thymine, respectively, adenosine deaminase capable of converting adenine to hypoxanthine, guanosine deaminase capable of converting guanine to xanthine and the like. As cytidine deaminase, more preferred is APOBEC. In human, APOBEC includes APOBEC1, APOBEC2, APOBEC3 (e.g., APOBEC3A, APOBEC3B, APOBEC3C, APOBEC3D(APOBEC3E), APOBEC3F, APOBEC3G, APOBEC3H), APOBEC4, activation-induced cytidine deaminase (AID) which is an enzyme that introduces a mutation into an immunoglobulin gene in the acquired immunity of vertebrate and the like.

The DNA modifying enzyme-binding module used in the method of the present invention is not particularly limited as long as it can bind to the cell-endogenous DNA modifying enzyme mentioned above. Examples thereof include antibody, peptide aptamer, nucleic acid aptamer against the target DNA modifying enzyme, proteins that bind to other DNA modifying enzymes and the like. The DNA modifying enzyme-binding module can be appropriately selected according to the type of target DNA modifying enzyme. As these DNA modifying enzyme-binding modules, those known to bind to the target DNA modifying enzyme may be used, or molecules produced by the method described below may be used. The DNA encoding the DNA modifying enzyme-binding module can be appropriately produced based on the information of the amino acid sequence, nucleic acid sequence of the object DNA modifying enzyme-binding module.

The antibody used in the method of the present invention may be either a polyclonal antibody or a monoclonal antibody, and the antibody also encompasses antibody fragments (e.g., F(ab′)₂, Fab′, Fab, Fv, scFv etc.). The antibody can be produced by a well-known immunological method. Peptide aptamer is an aptamer composed of an amino acid and is a peptide molecule that can bind to a specific target molecule, similar to antibodies. The peptide aptamer can be screened for or produced based on a phage display method and a cell surface layer display method (e.g., Whaley, S. R., et al., (2000), Nature, 405, 665-668). The nucleic acid aptamer is an aptamer constituted of RNA, DNA, modified nucleotide or a mixture thereof. The aptamer can be screened for or produced according to well-known methods (e.g., Ellington et al., (1990), Nature, 346,818-822; Tuerk et al., (1990) Science, 249, 505-510).

Examples of the protein that binds to a DNA modifying enzyme include, but are not limited to, Vif (Virion Infectivity Factor) of human immunodeficiency virus (HIV) and monkey immunodeficiency virus (SIVmac) known to bind to APOBEC (particularly, APOBEC3), Bet (Bromodomain and extra-terminal) protein of foamy virus, TopoIIβ (Topoisomerase 2-beta), IQGAP2, ZNF335 (aka: NIF1), CD81, MLL, C-terminal (196th-384th amino acid residues) of APOBEC3G (e.g., Schumacher, April Jean, Ph.D., UNIVERSITY OF MINNESOTA, (2008) 199, pages; 3313466), fragments of these (in the following, unless otherwise specified, protein encompasses fragments thereof) and the like. These proteins may be altered (altered protein is sometimes referred to as a “variant” of protein). For example, when Vif is used, since Vif is known to bind to an E3 ubiquitin ligase complex and promote proteolysis of APOBEC3 (e.g., Stanley et al. (2008) Journal of virology, 8656-8663; Guo et al. (2014) Nature, 55, 229-233), it is preferable to apply alteration that causes lack of bindability to proteins other than APOBEC3. Examples of such alteration include deletion of several (e.g., 11, 10, 9, 8, 7 etc.) amino acids in the N terminal of Vif protein (refseq No. AAF20197) and substitution of the 145th leucine residue with other amino acid residue (e.g., alanine residue) and the like, but they are not limited to these alterations. Even when a protein other than Vif is used, it can be appropriately modified based on the function of the protein, binding site with the target molecule, three-dimensional structure, and the like. The above-mentioned protein fragment is not particularly limited as long as it has a binding region to the DNA modifying enzyme. For example, a fragment excluding a region other than the binding region to the DNA modifying enzyme (e.g., region having protein catalytic activity) can be mentioned. Specific examples of such fragment include a peptide composed of the 452nd-591st amino acid residues of TopoIIβ (refseq No.: NP 001059), a peptide composed of the 466th—547th amino acid residues of IQGAP2 (refseq No.: NP 006624), a peptide composed of the 745th—893rd amino acid residues of ZNF335 (refseq No.: NP 071378) and the like. These are mere examples, and those skilled in the art can appropriately design fragments. As shown in the below-mentioned Examples, the targeted site is also altered when IQGAP2 and ZNF335 are combined (Table 2). Accordingly, proteins that bind to the aforementioned DNA modifying enzyme can also be used in combination.

In the present invention, the “base excision repair” is one of the DNA repair mechanisms of living organisms, and means a mechanism for repairing damages of bases by cutting off damaged parts of the bases by enzymes and rejoining them. Excision of damaged bases is performed by DNA glycosylase, which is an enzyme that hydrolyzes the N-glycoside linkage of DNA. An abasic site (apurinic/apyrimidic (AP) site) resulting from the abasic reaction by the enzyme is treated by an enzyme at the downstream of the base excision repair (BER) pathway such as AP endonuclease, DNA polymerase, DNA ligase and the like. Examples of such gene or protein involved in the BER pathway include, but are not limited to, UNG (NM 003362), SMUG1 (NM 014311), MBD4 (NM 003925), TDG (NM 003211), OGG1 (NM 002542), MYH (NM 012222), NTHL1 (NM 002528), MPG (NM 002434), NEIL1 (NM 024608), NEIL2 (NM 145043), NEIL3 (NM 018248), APE1 (NM 001641), APE2 (NM 014481), LIG3 (NM 013975), XRCC1 (NM 006297), ADPRT (PARP1) (NM 0016718), ADPRTL2 (PARP2) (NM 005484) and the like (parentheses indicate refseq number in which the base sequence information of each gene (cDNA) is registered).

In the present invention, the “base excision repair inhibitor” means a substance that inhibits any stage of the above-mentioned BER pathway, or a substance that eventually inhibits BER by inhibiting the expression of molecules mobilized in the BER pathway. While the base excision repair inhibitor to be used in the present invention is not particularly limited as long as it consequently inhibits BER, from the aspect of efficiency, an inhibitor of DNA glycosylase located at the upstream of the BER pathway is preferable. Examples of the inhibitor of DNA glycosylase to be used in the present invention include, but are not limited to, a thymine DNA glycosylase inhibitor, an uracil DNA glycosylase inhibitor, an oxoguanine DNA glycosylase inhibitor, an alkylguanine DNA glycosylase inhibitor and the like. For example, when the target enzyme of a DNA modifying enzyme-binding module is cytidine deaminase, it is suitable to use a uracil DNA glycosylase inhibitor to inhibit repair of U:G or G:U mismatch of DNA generated by mutation.

Examples of such uracil DNA glycosylase inhibitor include, but are not limited to, a uracil DNA glycosylase inhibitor (Ugi) derived from Bacillus subtilis bacteriophage, PBS1, and a uracil DNA glycosylase inhibitor (Ugi) derived from Bacillus subtilis bacteriophage, PBS2 (Wang, Z., and Mosbaugh, D. W. (1988) J. Bacteriol. 170, 1082-1091). The above-mentioned inhibiter of the repair of DNA mismatch can be used in the present invention. Particularly, Ugi derived from PBS2 is also known to have an effect of making it difficult to cause mutation, cleavage and recombination other than T from C on DNA, and thus the use of Ugi derived from PBS2 is suitable.

As mentioned above, in the base excision repair (BER) mechanism, when a base is excised by DNA glycosylase, AP endonuclease puts a nick in the abasic site (AP site), and exonuclease completely excises the AP site. When the AP site is excised, DNA polymerase produces a new base by using the base of the opposing strand as a template, and DNA ligase finally seals the nick to complete the repair. Mutant AP endonuclease that has lost the enzyme activity but maintains the binding capacity to the AP site is known to competitively inhibit BER. Therefore, these mutation AP endonucleases can also be used as the base excision repair inhibitor in the present invention. While the derivation of the mutant AP endonuclease is not particularly limited, for example, AP endonucleases derived from Escherichia coli, yeast, mammal (e.g., human, mouse, swine, bovine, horse, monkey etc.) and the like can be used. For example, UniprotKB No. P27695 can be referred to for the amino acid sequence of human Apel. Examples of the mutant AP endonuclease that has lost the enzyme activity but maintains the binding capacity to the AP site include proteins having mutated activity site and mutated Mg (cofactor)-binding site. For example, E96Q, Y171A, Y171F, Y171H, D210N, D210A, N212A and the like can be mentioned for human Apel.

A target nucleotide sequence in a DNA to be recognized by the nucleic acid sequence-recognizing module in the complex of the present invention is not particularly limited as long as the module specifically binds to, and may be any sequence in the DNA. The length of the target nucleotide sequence only needs to be sufficient for specific binding of the nucleic acid sequence-recognizing module. For example, it is not less than 12 nucleotides, preferably not less than 15 nucleotides, more preferably not less than 18 nucleotides, according to the size of the target DNA. While the upper limit of the length is not particularly limited, it is preferably not more than 25 nucleotides, more preferably not more than 22 nucleotides.

As the nucleic acid sequence-recognizing module in the complex of the present invention, CRISPR-Cas system wherein at least one DNA cleavage ability of Cas is inactivated (hereinafter to be also referred to as “CRISPR-mutant Cas”), zinc finger motif, TAL effector and PPR motif and the like, as well as a fragment containing a DNA binding domain of a protein that specifically binds to DNA, such as restriction enzyme, transcription factor, RNA polymerase and the like, and free of a DNA double strand cleavage ability and the like can be used, but the module is not limited thereto. Preferably, CRISPR-mutant Cas, zinc finger motif, TAL effector, PPR motif and the like can be mentioned.

A zinc finger motif is constituted by linkage of 3-6 different Cys2His2 type zinc finger units (1 finger recognizes about 3 bases), and can recognize a target nucleotide sequence of 9-18 bases. A zinc finger motif can be produced by a known method such as Modular assembly method (Nat Biotechnol (2002)20: 135-141), OPEN method (Mol Cell (2008) 31: 294-301), CoDA method (Nat Methods (2011) 8: 67-69), Escherichia coli one-hybrid method (Nat Biotechnol (2008)26:695-701) and the like. The above-mentioned patent document 1 can be referred to as for the detail of the zinc finger motif production.

A TAL effector has a module repeat structure with about 34 amino acids as a unit, and the 12th and 13th amino acid residues (called RVD) of one module determine the binding stability and base specificity. Since each module is highly independent, TAL effector specific to a target nucleotide sequence can be produced by simply connecting the module. For TAL effector, a production method utilizing an open resource (REAL method (Curr Protoc Mol Biol (2012) Chapter 12: Unit 12.15), FLASH method (Nat Biotechnol (2012) 30: 460-465), and Golden Gate method (Nucleic Acids Res (2011) 39: e82) etc.) have been established, and a TAL effector for a target nucleotide sequence can be designed comparatively conveniently. The above-mentioned patent document 2 can be referred to as for the detail of the production of TAL effector.

PPR motif is constituted such that a particular nucleotide sequence is recognized by a continuation of PPR motifs each consisting of 35 amino acids and recognizing one nucleic acid base, and recognizes a target base only by 1, 4 and ii(−2) amino acids of each motif. Motif constituent has no dependency, and is free of interference of motifs on both sides. Therefore, like TAL effector, a PPR protein specific to the target nucleotide sequence can be produced by simply connecting PPR motifs. The above-mentioned patent document 4 can be referred to as for the detail of the production of PPR motif.

When a fragment of restriction enzyme, transcription factor, RNA polymerase and the like is used, since the DNA binding domains of these proteins are well known, a fragment containing the domain and free of a DNA double strand cleavage ability can be easily designed and constructed.

Any of the above-mentioned nucleic acid sequence-recognizing modules can be provided as a fusion protein with the above-mentioned DNA modifying enzyme-binding module when it is a protein, or a protein binding domain such as SH3 domain, PDZ domain, GK domain, GB domain and the like and a binding partner thereof may be fused with a nucleic acid sequence-recognizing module and a DNA modifying enzyme-binding module, respectively, and provided as a protein complex via an interaction of the domain and a binding partner thereof. Alternatively, a nucleic acid sequence-recognizing module and a DNA modifying enzyme-binding module may be each fused with intein, and they can be linked by ligation after protein synthesis.

The complex of the present invention containing a complex (including fusion protein) wherein a nucleic acid sequence-recognizing module and DNA modifying enzyme-binding module are bonded is desirably contacted with a DNA (e.g., genomic DNA) by introducing a nucleic acid encoding the complex into a cell having the object DNA.

Therefore, the nucleic acid sequence-recognizing module and the DNA modifying enzyme-binding module are preferably prepared as a nucleic acid encoding a fusion protein thereof, or in a form capable of forming a complex in a host cell after translation into a protein by utilizing a binding domain, intein and the like, or as a nucleic acid encoding each of them. The nucleic acid here may be a DNA or an RNA, preferably DNA. When it is a DNA, it is preferably a double-stranded DNA, and provided in the form of an expression vector disposed under regulation of a functional promoter in a host cell.

The complex of the present invention wherein a nucleic acid sequence-recognizing module and a DNA modifying enzyme-binding module are bonded permits DNA editing with low toxicity is possible, and the genetic alteration method of the present invention can be applied to a wide range of biological materials. Therefore, the cells to be introduced with nucleic acid encoding nucleic acid sequence-recognizing module and/or DNA modifying enzyme-binding module can encompass cells of any species, from bacterium of Escherichia coli and the like which are prokaryotes, cells of microorganism such as yeast and the like which are lower eucaryotes, to cells of vertebrate including mammals such as human and the like, and cells of higher eukaryote such as insect, plant and the like.

A DNA encoding a nucleic acid sequence-recognizing module such as zinc finger motif, TAL effector, PPR motif and the like can be obtained by any method mentioned above for each module.

A DNA encoding a sequence-recognizing module of restriction enzyme, transcription factor, RNA polymerase and the like can be cloned by, for example, synthesizing an oligoDNA primer covering a region encoding a desired part of the protein (i.e., part containing DNA binding domain) based on the cDNA sequence information thereof, and amplifying by the RT-PCR method using, as a template, the total RNA or mRNA fraction prepared from the protein-producing cells.

A DNA encoding DNA modifying enzyme-binding module, DNA modifying enzyme inducer or base excision repair inhibitor can also be cloned similarly by synthesizing an oligoDNA primer based on the cDNA sequence information of the protein and the like to be used, and amplifying by the RT-PCR method using, as a template, the total RNA or mRNA fraction prepared from the protein and the like. For example, when Vif of HIV is used as a DNA modifying enzyme-binding module, a DNA encoding the protein can be cloned by designing suitable primers for the upstream and downstream of CDS based on the cDNA sequence (accession No. AF200477) registered in the NCBI database, and performing cloning according to the RT-PCR method from RNA extracted from a cell infected with HIV.

The cloned DNA may be directly, or after digestion with a restriction enzyme when desired, or after addition of a suitable linker and/or a nuclear localization signal (each organelle localization signal when the object DNA is mitochondria or chloroplast DNA), ligated with a DNA encoding a nucleic acid sequence-recognizing module to prepare a DNA encoding a fusion protein. When a nucleic acid sequence-recognizing module and a DNA modifying enzyme-binding module are expressed as a fusion protein, for example, a nuclear localization signal can be added to the both terminals of the fusion protein, or between the nucleic acid sequence-recognizing module and the DNA modifying enzyme-binding module. The nuclear localization signal is not particularly limited and, for example, SV40-derived nuclear localization signal (e.g., SEQ ID NO: 7, SEQ ID NO: 9) can be mentioned.

Alternatively, a DNA encoding a nucleic acid sequence-recognizing module, and a DNA encoding a DNA modifying enzyme-binding module may be each fused with a DNA encoding a binding domain or a binding partner thereof, or respective DNAs may be fused with a DNA encoding a separation intein, whereby the nucleic acid sequence-recognizing module and the DNA modifying enzyme-binding module are translated in a host cell to form a complex. In these cases, a linker and/or a nuclear localization signal can be linked to a suitable position of respective DNAs when desired.

A DNA encoding nucleic acid sequence-recognizing module and a DNA encoding DNA modifying enzyme-binding module (and a DNA encoding DNA modifying enzyme inducer when cells are stimulated by introducing and expressing DNA encoding the inducer in the cell; hereinafter the same when indicated in parentheses) can be obtained by chemically synthesizing the DNA strand, or by connecting synthesized partly overlapping oligoDNA short strands by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (http://www.kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency. For example, when the host cell is a human cell, a nucleic acid sequence-recognizing module and/or a sequence encoding a DNA modifying enzyme-binding module which are/is optimized for use of human codon can be used. A DNA encoding a base excision repair inhibitor can also be constructed similarly.

An expression vector containing a DNA encoding nucleic acid sequence-recognizing module and/or a DNA encoding DNA modifying enzyme-binding module (and/or a DNA encoding a DNA modifying enzyme inducer) can be produced, for example, by linking the DNA to the downstream of a promoter in a suitable expression vector. Furthermore, the aforementioned expression vector can also be produced including a DNA encoding a base excision repair inhibitor.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as Aphage and the like; insect virus vectors such as baculovirus and the like (e.g., BmNPV, AcNPV); animal virus vectors such as retrovirus, vaccinia virus, adenovirus and the like, and the like are used.

As the promoter, any promoter appropriate for a host to be used for gene expression can be used. In a conventional method accompanying DSB, since the survival rate of the host cell sometimes decreases markedly due to the toxicity, it is desirable to increase the number of cells by the start of the induction by using an inductive promoter. When an enzyme unaccompanied by DSB is induced as a cell-endogenous DNA modifying enzyme, since sufficient cell proliferation can also be expected by expressing the complex of the present invention, a constituent promoter can also be used without limitation.

For example, when the host is an animal cell, SRα promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like are used. Of these, CMV promoter, SRα promoter and the like are preferable.

When the host is Escherichia coli, trp promoter, lac promoter, recA promoter, λP_{L} promoter, 1pp promoter, T7 promoter and the like are preferable.

When the host is genus Bacillus, SPOT promoter, SPO2 promoter, penP promoter and the like are preferable.

When the host is a yeast, Gal1/10 promoter, PHO5 promoter, PGK promoter, GAP promoter, ADH promoter and the like are preferable.

When the host is an insect cell, polyhedrin promoter, P10 promoter and the like are preferable.

When the host is a plant cell, CaMV35S promoter, CaMV19S promoter, NOS promoter and the like are preferable.

When desired, the expression vector can contain a terminator (e.g., NOS terminator, Pisum sativum rbcS3A terminator, heat shock protein (HSP)17.3 terminator etc.), a translation enhancer (e.g., rice derived from alcoholdehydrogenase 5′ untranslated region (Os ADH-5′UTR), CaMV or tobacco mosaic virus (TMV)-derived Ω sequence etc.), a 3′ regulatory region (e.g., rice derived from actin gene (Act1)3′UTR etc.), poly A added signal, a selection marker of a drug resistance gene (e.g., G418 resistance gene (nPtII), hygromycin resistance gene (hpt) etc.) and the like.

An RNA encoding a nucleic acid sequence-recognizing module and/or an RNA encoding a DNA modifying enzyme-binding module (and/or an RNA encoding DNA modifying enzyme inducer) can be prepared by, for example, transcription to mRNA in vitro transcription system known per se by using the above-mentioned expression vector containing a DNA encoding nucleic acid sequence-recognizing module and/or a DNA encoding DNA modifying enzyme-binding module (and/or a DNA encoding DNA modifying enzyme inducer) as a template. RNA encoding a base excision repair inhibitor can be prepared similarly.

A complex of a nucleic acid sequence-recognizing module and a DNA modifying enzyme-binding module can be intracellularly expressed by introducing an expression vector containing a DNA encoding a nucleic acid sequence-recognizing module and/or a DNA modifying enzyme-binding module into a host cell, and culturing the host cell.

As the host, genus Escherichia, genus Bacillus, yeast, insect cell, insect, animal cell and the like are used.

As the genus Escherichia, Escherichia coli K12·DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], Escherichia coli C600 [Genetics, 39, 440 (1954)] and the like are used.

As the genus Bacillus, Bacillus subtilis MI114 [Gene, 24, 255 (1983)], Bacillus subtilis 207-21 [Journal of Biochemistry, 95, 87 (1984)] and the like are used.

As the yeast, Saccharomyces cerevisiae AH22, AH22R—, NA87-11A, DKD-5D, 20B-12, Schizosaccharomyces pombe NCYC1913, NCYC2036, Pichia pastoris KM71 and the like are used.

As the insect cell when the virus is AcNPV, cells of cabbage armyworm larva-derived established line (Spodoptera frugiperda cell; Sf cell), MG1 cells derived from the mid-intestine of Trichoplusia ni, High Five™ cells derived from an egg of Trichoplusia ni, Mamestra brassicae-derived cells, Estigmena acrea-derived cells and the like are used. When the virus is BmNPV, cells of Bombyx mori-derived established line (Bombyx mori N cell; BmN cell) and the like are used as insecT cells. As the Sf cell, for example, Sf9 cell (ATCC CRL1711), Sf21 cell [all above, In Vivo, 13, 213-217 (1977)] and the like are used.

As the insect, for example, larva of Bombyx mori, Drosophila, cricket and the like are used [Nature, 315, 592 (1985)].

As the animal cell, cell lines such as monkey COS-7 cell, monkey Vero cell, Chinese hamster ovary (CHO) cell, dhfr gene-deficient CHO cell, mouse L cell, mouse AtT-20 cell, mouse myeloma cell, rat GH3 cell, human fetal kidney-derived cells (e.g., HEK293 cell), cell derived from human liver cancer (e.g., HepG2), human FL cell and the like, pluripotent stem cells such as iPS cell, ES cell and the like of human and other mammals, and primary cultured cells prepared from various tissues are used. Furthermore, zebrafish embryo, Xenopus oocyte and the like can also be used.

As the plant cell, suspend cultured cells, callus, protoplast, leaf segment, root segment and the like prepared from various plants (e.g., grain such as rice, wheat, corn and the like, product crops such as tomato, cucumber, egg plant and the like, garden plants such as carnation, Eustoma russellianum and the like, experiment plants such as tobacco, Arabidopsis thaliana and the like, and the like) are used.

All the above-mentioned host cells may be haploid (monoploid), or polyploid (e.g., diploid, triploid, tetraploid and the like). In the conventional mutation introduction methods, mutation is, in principle, introduced into only one homologous chromosome to produce a hetero gene type. Therefore, desired phenotype is not expressed unless dominant mutation occurs, and homozygousness inconveniently requires labor and time. In contrast, according to the present invention, since mutation may be introduced into any allele on the homologous chromosome in the genome when the target DNA is altered by the method of the present invention using a CRISPR-mutation Cas including nucleic acid sequence-recognizing module, desired phenotype can be expressed in a single generation even in the case of recessive mutation, which can solve the problem of the conventional method.

An expression vector can be introduced by a known method (e.g., lysozyme method, competent method, PEG method, CaCl₂ coprecipitation method, electroporation method, the microinjection method, the particle gun method, lipofection method, Agrobacterium method and the like) according to the kind of the host.

Escherichia coli can be transformed according to the methods described in, for example, Proc. Natl. Acad. Sci. USA, 69, 2110 (1972), Gene, 17, 107 (1982) and the like.

The genus Bacillus can be introduced into a vector according to the methods described in, for example, Molecular & General Genetics, 168, 111 (1979) and the like.

A yeast can be introduced into a vector according to the methods described in, for example, Methods in Enzymology, 194, 182-187 (1991), Proc. Natl. Acad. Sci. USA, 75, 1929 (1978) and the like.

An insect cell and an insect can be introduced into a vector according to the methods described in, for example, Bio/Technology, 6, 47-55 (1988) and the like.

An animal cell can be introduced into a vector according to the methods described in, for example, Cell Engineering additional volume 8, New Cell Engineering Experiment Protocol, 263-267 (1995) (published by Shujunsha), and Virology, 52, 456 (1973).

A cell introduced with a vector can be cultured according to a known method according to the kind of the host.

For example, when Escherichia coli or genus Bacillus is cultured, a liquid medium is preferable as a medium to be used for the culture. The medium preferably contains a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is preferably about 5—about 8.

As a medium for culturing Escherichia coli, for example, M9 medium containing glucose, casamino acid [Journal of Experiments in Molecular Genetics, 431-433, Cold Spring Harbor Laboratory, New York 1972] is preferable. Where necessary, for example, agents such as 313-indolylacrylic acid may be added to the medium to ensure an efficient function of a promoter. Escherichia coli is cultured at generally about 15—about 43° C. Where necessary, aeration and stirring may be performed.

The genus Bacillus is cultured at generally about 30—about 40° C. Where necessary, aeration and stirring may be performed.

Examples of the medium for culturing yeast include Burkholder minimum medium [Proc. Natl. Acad. Sci. USA, 77, 4505 (1980)], SD medium containing 0.5% casamino acid [Proc. Natl. Acad. Sci. USA, 81, 5330 (1984)] and the like. The pH of the medium is preferably about 5—about 8. The culture is performed at generally about 20° C.—about 35° C. Where necessary, aeration and stirring may be performed.

As a medium for culturing an insect cell or insect, for example, Grace's Insect Medium [Nature, 195, 788 (1962)] containing an additive such as inactivated 10% bovine serum and the like as appropriate and the like are used. The pH of the medium is preferably about 6.2—about 6.4. The culture is performed at generally about 27° C. Where necessary, aeration and stirring may be performed.

As a medium for culturing an animal cell, for example, minimum essential medium (MEM) containing about 5—about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium is preferably about 6—about 8. The culture is performed at generally about 30° C.—about 40° C. Where necessary, aeration and stirring may be performed.

As a medium for culturing a plant cell, for example, MS medium, LS medium, B5 medium and the like are used. The pH of the medium is preferably about 5—about 8. The culture is performed at generally about 20° C.—about 30° C. Where necessary, aeration and stirring may be performed.

As mentioned above, a complex of a nucleic acid sequence-recognizing module and a DNA modifying enzyme-binding module, i.e., the complex of the present invention, can be expressed intracellularly.

An RNA encoding a nucleic acid sequence-recognizing module and/or DNA modifying enzyme-binding module can be introduced into a host cell by microinjection method, lipofection method and the like. RNA introduction can be performed once or repeated multiple times (e.g., 2-5 times) at suitable intervals.

When a complex of a nucleic acid sequence-recognizing module and a DNA modifying enzyme-binding module is expressed by an expression vector introduced into the cell, the nucleic acid sequence-recognizing module specifically recognizes and binds to a target nucleotide sequence in the DNA (e.g., genomic DNA) of interest. A DNA modifying enzyme-binding module linked to the nucleic acid sequence-recognizing module binds to a cell-endogenous DNA modifying enzyme induced by stimulation by a DNA modifying enzyme inducer, and DNA strand or base is modified in the targeted site (whole or partial target nucleotide sequence or the vicinity thereof) by the action of the DNA modifying enzyme.

When the target DNA is double stranded, modification of DNA occurs in the sense strand or antisense strand in the targeted site. When the modification of DNA is cleavage of DNA strand, various mutations are introduced during repair by the repair mechanism such as base excision repair (BER), nucleotide excision repair (NER), single strand cleavage repair, non-homologous end-joining (NHEJ), homologous recombination (HR) and the like. When the modification of DNA does not directly accompany cleavage of DNA strand, a mismatch or site free of base is produced in the double stranded DNA (AP moiety) (apurinic/apyrimidic (AP) site), mutations are introduced in the process of repairing same. For example, when a DNA modifying enzyme-binding module capable of binding to cytidine deaminase such as APOBEC and the like is used, cytosine on the sense strand or antisense strand at the targeted site is converted to uracil to cause U:G or G:U mismatch). When the mismatch is not correctly repaired, and when repaired such that a base of the opposite strand forms a pair with a base of the converted strand (T-A or A-T in the above-mentioned example), or when other nucleotide is further substituted (e.g., U→A, G) or when one to several dozen bases are deleted or inserted during repair, various mutations are introduced. For example, when a DNA modifying enzyme-binding module capable of binding to DNA glycosylase is used, base excision reaction occurs in the sense strand or antisense strand of the targeted site, and an abasic site (AP site) is produced in one of the strands of the double stranded DNA. Then, the base excision repair (BER) system in the cell operates, AP endonuclease first recognizes the AP site and cleaves the phosphoric acid bond in one of DNA strand, and exonuclease removes nucleotide subjected to base excision. Then, DNA polymerase inserts a new nucleotide by using the opposing strand DNA as a template and finally DNA ligase repairs the joint. Various mutations are introduced by a repair miss occurring at any stage of this BER.

As for zinc finger motif, production of many actually functionable zinc finger motifs is not easy, since production efficiency of a zinc finger that specifically binds to a target nucleotide sequence is not high and selection of a zinc finger having high binding specificity is complicated. While TAL effector and PPR motif have a high degree of freedom of target nucleic acid sequence recognition as compared to zinc finger motif, a problem remains in the efficiency since a large protein needs to be designed and constructed every time according to the target nucleotide sequence.

In contrast, since the CRISPR-Cas system recognizes the object DNA sequence by a guide RNA complementary to the target nucleotide sequence, any sequence can be targeted by simply synthesizing an oligoDNA capable of specifically forming a hybrid with the target nucleotide sequence.

Therefore, in a more preferable embodiment of the present invention, a CRISPR-Cas system wherein at least one DNA cleavage ability of Cas effector protein is inactivated (CRISPR-mutant Cas) is used as a nucleic acid sequence-recognizing module.

The nucleic acid sequence-recognizing module of the present invention using CRISPR-mutant Cas is provided as a complex of a CRISPR-RNA (crRNA) containing a sequence complementary to the target nucleotide sequence and, where necessary, trans-activating RNA (tracrRNA) necessary for recruiting mutant Cas effector protein (when tracrRNA is necessary, possibly provided as chimeric RNA with crRNA) and mutant Cas effector protein. An RNA molecule consisting of crRNA alone or a chimeric RNA of crRNA and tracrRNA that constitutes a nucleic acid sequence-recognizing module in combination with a mutant Cas effector protein is collectively referred to as “guide RNA”. When a nucleic acid aptamer is used as a DNA modifying enzyme-binding module, the nucleic acid aptamer is desirably bonded to the guide RNA. A nucleic acid in which a guide RNA and a nucleic acid aptamer are bonded can be produced by a known method (e.g., Mali et al., (2013), Nat Biotechnol, 31(9), 833-838).

While the Cas effector protein to be used in the present invention is not particularly limited as long as it an effector protein belonging to the class 2 CRISPR system capable of forming a complex with guide RNA and recognizing and binding to the target nucleotide sequence in the object gene and a protospacer adjacent motif (PAM) adjacent thereto, it is preferably Cas9 or Cpf1. Examples of Cas9 include, but are not limited to, Cas9 (SpCas9) derived from Streptococcus pyogenes; PAM sequence (5″→3″direction; hereinafter the same) NGG (N is A, G, T or C, hereinafter the same)), Cas9 (StCas9; PAM sequence NNAGAAW) derived from Streptococcus thermophilus, Cas9 (MmCas9; PAM sequence NNNNGATT) derived from Neisseria meningitidis and the like. Preferred is SpCas9 with less restriction by PAM (substantially 2 bases, and can target theoretically any site on the genome). Examples of the Cpf1 include, but are not limited to, Cpf1 derived from Francisella novicida (FnCpf1; PAM sequence TTN), Cpf1 derived from Acidaminococcus sp. (AsCpf1; PAM sequence TTTN), Cpf1 derived from Lachnospiraceae bacterium (LbCpf1; PAM sequence TTTN) and the like. As a mutant Cas effector protein (hereinafter sometimes to be abbreviated as “mutation Cas”) to be used in the present invention, any of Cas effector protein wherein the cleavage ability of the both strands of the double-stranded DNA is inactivated and one having nickase activity wherein at least one cleavage ability of one strand alone is inactivated can be used. For example, in the case of SpCas9, a D10A mutant in which the 10th Asp residue is converted to an Ala residue and lacking cleavage ability of a strand opposite to the strand forming a complementary strand with a guide RNA (thus having nickase activity for a strand forming complementary strand with guide RNA), or H840A mutant in which the 840th His residue is converted to an Ala residue and lacking cleavage ability of a strand forming a complementary strand to guide RNA (thus having nickase activity for a strand forming complementary strand with guide RNA, or a double mutant thereof (dCas9) can be used. In the case of FnCpf1, a variant in which the 917th Asp residue is converted to Ala residue (D917A) or the 1006th Glu residue is converted to Ala residue (E1006A), and lacking cleavage ability of both strands can be used. As long as at least one of the strands of double stranded DNA lacks cleavage ability, other mutant Cas can also be used similarly.

The DNA modifying enzyme-binding module is provided as a complex with mutant Cas by a method similar to the coupling scheme with the above-mentioned zinc finger and the like. Alternatively, a DNA modifying enzyme-binding module and mutant Cas can also be bound by utilizing RNA aptamers MS2F6, PP7 and the like and RNA scaffold by binding proteins thereto. The targeting sequence in the guide RNA forms a complementary strand with the target nucleotide sequence, mutant Cas is recruited by the tracrRNA attached and mutant Cas recognizes PAM. One or both DNAs cannot be cleaved and, due to the action of the DNA modifying enzyme-binding module linked to the mutant Cas, base conversion occurs in the targeted site (appropriately adjusted within several hundred bases including whole or partial target nucleotide sequence) and a mismatch occurs in the double stranded DNA. When the mismatch is not correctly repaired, and when repaired such that a base of the opposite strand forms a pair with a base of the converted strand, or when other nucleotide is further converted or when one to several dozen bases are deleted or inserted during repair, various mutations are introduced.

When CRISPR-mutant Cas is used as a nucleic acid sequence-recognizing module, similar to when zinc finger and the like are used as a nucleic acid sequence-recognizing module, a nucleic acid sequence-recognizing module and a DNA modifying enzyme-binding module are desirably introduced, in the form of a nucleic acid (preferably DNA) encoding same, into a cell having a DNA of interest.

A DNA encoding Cas effector protein (e.g., Cas9, Cpf1) can be cloned by a method similar to the above-mentioned method for a DNA encoding a DNA modifying enzyme-binding module, from a cell producing the protein. A mutant Cas can be obtained by introducing a mutation to convert an amino acid residue of the part important for the DNA cleavage activity (e.g., 10th Asp residue and 840th His residue for SpCas9, 917th Asp residue, 1006th Glu residue and 1255th Asp residue for FnCpf1 and the like, though not limited thereto) to other amino acid, into a DNA encoding cloned Cas, by a site specific mutation induction method known per se. In addition, by constructing full-length DNA by chemical synthesis or in combination with PCR method or Gibson Assembly method, codons to be used can also be designed over the full-length CDS according to the host into which the DNA is introduced. For example, as SpCas9 DNA introduced with such mutation and using a codon suitable for expression in human cells, a DNA having the nucleotide sequence shown in SEQ ID NO: 4 can be mentioned.

The obtained a DNA encoding a mutant Cas and/or a DNA encoding a DNA modifying enzyme-binding module can be inserted into the downstream of a promoter of an expression vector similar to the one mentioned above, according to the host cell. As mentioned above, the expression vector can contain, when desired, selection markers such as terminator, translation enhancer, 3′ regulatory region, polyA addition signal, drug resistance gene and the like, and the like.

On the other hand, a DNA encoding guide RNA can be obtained by designing an oligoDNA sequence linking a coding sequence of crRNA sequence containing a nucleotide sequence complementary to the target nucleotide sequence (to be also referred to as “targeting sequence” in the present specification) (e.g., when FnCpf1 is recruited as Cas effector protein, crRNA containing SEQ ID NO: 10; AAUUUCUACUGUUGUAGAU at the 5′-side of the targeting sequence can be used, and underlined sequences form base pairs to take a stem-loop structure), or a crRNA coding sequence and, as necessary, a known tracrRNA coding sequence (e.g., as tracrRNA coding sequence when Cas is recruited as Cas9 effector protein, gttttagagctagaaatagcaagttaaaataaggctagtccgttatcaacttgaaaaagtggc accgagtcggtgc; SEQ ID NO: 11) and chemically synthesizing using a DNA/RNA synthesizer. When the target DNA is double stranded, crRNA sequence includes a nucleotide sequence complementary to a “targeted strand” of the target nucleotide sequence.

The “targeted strand” here means a strand forming a hybrid with crRNA of the target nucleotide sequence, and an opposite strand thereof that becomes single-stranded by hybridization to the targeted strand and crRNA is referred to as a “non-targeted strand”. Since the DNA modification reaction is generally assumed to frequently occur on a single stranded non-targeted strand, when the target nucleotide sequence is to be expressed by one of the strands (e.g., when PAM sequence is indicated, when positional relationship of target nucleotide sequence and PAM is shown etc.), it is represented by a sequence of the non-targeted strand.

While the length of the targeting sequence is not particularly limited as long as it can specifically bind to a target nucleotide sequence, for example, it is 15-30 nucleotides, preferably 18-25 nucleotides. The selection of the target nucleotide sequence is restricted by the presence of an adjacent PAM on the 3′-side (in the case of Cas9) or 5′-side (in the case of Cpf1) of the sequence. According to the finding in yeast and the like, in a system in which CRISPR-mutated Cas and cytidine deaminase are combined, C at a position within 7 nucleotides from the 5′-end thereof toward 3′ direction thereof is easily substituted irrespective of the length of the target nucleotide sequence. Therefore, by appropriately determining the length of the target nucleotide sequence (targeting sequence as a complementary strand thereof), the site of a base into which a mutation can be introduced may be shifted. As a result, restriction by PAM (NGG in SpCas9) may be removed at least partially, and the degree of freedom of mutation introduction is expected to be higher.

When Cas9 is used as a Cas effector protein, a targeting sequence can be designed, for example, using a guide RNA design website open to public (CRISPR Design Tool, CRISPRdirect etc.) by listing up 20 mer sequences having PAM (e.g., NGG in the case of SpCas9) adjacent to the 3′-side from the CDS sequences of the object gene, and selecting a sequence that causes an amino acid change in the protein encoded by the target gene when C within 7 nucleotides from the 5′ end thereof toward 3′ direction is converted to T. Furthermore, a sequence having C that similarly causes, when the length of the targeting sequence is changed, for example, within the range of 18-25 nucleotides, an amino acid change by base conversion to T within 7 nucleotides from the 5′ end thereof toward 3′ direction is selected. A candidate sequence having a small number of off-target sites in the genome of the host can be used as a targeting sequence. When the guide RNA design software to be used does not have a function to search off-target sites of the genome of the host, for example, off-target sites can be searched by applying a Blast search to the genome of the host, for example, 8-12 nucleotides on the 3′-side of the candidate sequence (seed sequence with high discrimination ability of target nucleotide sequence).

A DNA encoding guide RNA (e.g., crRNA or crRNA-tracrRNA chimera) can be obtained by designing an oligoDNA sequence linking a sequence complementary to the target strand of the target nucleotide sequence and a known tracrRNA sequence (when Cas9 is recruited) or a direct repeat sequence of crRNA (when Cpf1 is recruited) and chemically synthesizing using a DNA/RNA synthesizer. While a DNA encoding guide RNA can also be inserted into an expression vector similar to the one mentioned above, as the promoter, pol III system promoter (e.g., SNR6, SNR52, SCR1, RPR1, U3, U6, H1 promoter etc.) and terminator (e.g., T₆ sequence; tttttt etc.) are preferably used.

DNA encoding mutant Cas, DNA encoding DNA modifying enzyme-binding module, a DNA encoding guide RNA can be introduced into a host cell by a method similar to the above, according to the host.

In genome editing using a complex of deaminase and a nucleic acid sequence-recognizing module (hereinafter sometimes to be referred to as “Target AID”) (patent document 5), the present inventors compared the effects of two kinds of mutant Cas having nickase activity of cleaving different strand and reported that mutated sites gathered near the center of the target nucleotide sequence in one of them and various mutations were randomly introduced into region of several hundred bases from the target nucleotide sequence in the other, and thus, similar effects can also be expected in the present invention. Therefore, by selecting a strand to be cleaved by the nickase, a mutation can be introduced into a particular nucleotide or nucleotide region at a pinpoint, or various mutations can be randomly introduced into a comparatively wide range, which can be property adopted according to the object. For example, when the former technique is applied to gene disease iPS cell, an agent for cell transplantation therapy with a reduced risk of rejection by repairing the mutation of the pathogenic gene in iPS cells prepared from the patient's own cells and then differentiating them into the desired somatic cells can be produced.

In Target AID, the present inventors also confirmed using a budding yeast that when sequence-recognizing modules are produced corresponding to the adjacent multiple target nucleotide sequences, and simultaneously used, the mutation introduction efficiency drastically increases than using a single nucleotide sequence as a target, and similar effects can also be expected in the present invention. When the target DNA a double stranded DNA, it can occur both when the target nucleotide sequences are in the same direction (i.e., targeted strands are on the same strand), and when they are opposed (i.e., both strands of double stranded DNA are targeted strands).

In addition, modification of multiple DNA regions at completely different positions as targets can also be performed. Therefore, in one preferable embodiment of the present invention, two or more kinds of nucleic acid sequence-recognizing modules that specifically bind to different target nucleotide sequences (which, when target DNA is cell-endogenous DNA, may be present in one object gene, or two or more different object genes) can be used. In this case, each one of these nucleic acid sequence-recognizing modules and a DNA modifying enzyme-binding module form a complex. Here, a common DNA modifying enzyme-binding module can be used. For example, when CRISPR-Cas system is used as a nucleic acid sequence-recognizing module, a common complex of Cas effector protein and DNA modifying enzyme-binding module (including fusion protein) is used, and two or more guide RNAs containing two or more crRNAs that respectively form a complementary strand with a different target nucleotide sequence are produced and can be used as guide RNA. On the other hand, when zinc finger motif, TAL effector and the like are used as nucleic acid sequence-recognizing modules, for example, a DNA modifying enzyme-binding module can be fused with a nucleic acid sequence-recognizing module that specifically binds to a different target nucleotide.

To express the complex of the present invention in a host cell, as mentioned above, an expression vector containing a DNA encoding a nucleic acid sequence-recognizing module and a DNA encoding a DNA modifying enzyme-binding module (both DNAs may be on separate vectors or a single vector) or RNAs encoding respective modules are introduced into a host cell. For efficient introduction of mutation, it is desirable to maintain an expression of the complex of the present invention at a given level or above for not less than a given period. From such aspect, it is ensuring to introduce an expression vector (e.g., plasmid etc.) autonomously replicatable in a host cell. However, since the plasmid etc. are foreign DNAs, they are preferably removed rapidly after successful introduction of mutation. Therefore, though subject to change depending on the kind of host cell and the like, for example, the introduced plasmid is desirably removed from the host cell after a lapse of 6 hr—2 days from the introduction of an expression vector by using various plasmid removal methods well known in the art.

Examples of the means for removing foreign DNA incorporated into the host genomic DNA include a method using a Cre-loxP system, a method using transposon and the like.

Alternatively, as long as expression of the complex of the present invention, which is sufficient for the introduction of mutation, is obtained, it is preferable to introduce mutation into the object DNA by transient expression by using an expression vector or RNA without autonomous replicatability in a host cell (e.g., vector etc. lacking replication origin that functions in host cell and/or gene encoding protein necessary for replication).

Alternatively, editing of the host DNA can be realized efficiently while avoiding the risk of off-target action by causing a DNA modification reaction in a desired stage, and transiently expressing the complex of the present invention in a host cell for a period necessary for fixing the alteration of the targeted site. While a period necessary for the DNA modification reaction and fixing the alteration of the targeted site can be appropriately determined similarly to the above-mentioned period for stimulating the cells with a DNA modifying enzyme inducer. The expression induction period of the a nucleic acid encoding the complex of the present invention may be extended beyond the above-mentioned period as long as the host cell is free of unpreferable side effects.

As a means for transiently expressing the complex of the present invention at a desired stage for a desired period, a method including producing a construct (expression vector) containing a DNA encoding the complex [i.e., DNA encoding nucleic acid sequence-recognizing module (DNA encoding a guide RNA and DNA encoding a mutant Cas in the CRISPR-Cas system), and DNA encoding DNA modifying enzyme-binding module (in the CRISPR-Cas system, a DNA encoding a DNA modifying enzyme-binding module can be linked to a DNA encoding a mutant Cas or a DNA encoding a guide RNA, respectively, depending on whether the module is a protein or RNA)] in a form permitting control of the expression period of the complex and introducing same into the host cell can be mentioned. The “form capable of controlling the expression period” is specifically, for example, a DNA encoding the complex of the present invention placed under regulation of an inducible regulatory region. While the “inducible regulatory region” is not particularly limited, it is, for example, an operon of a temperature sensitive (ts) mutation repressor and an operator regulated thereby in microbial cells such as bacterium (e.g., Escherichia coli), yeast and the like. Examples of the ts mutation repressor include, but are not limited to, ts mutation of Aphage-derived cI repressor. In the case of Aphage cI repressor (ts), it is bound to an operator to suppress expression of gene in the downstream at not more than 30° C. (e.g., 28° C.). At a high temperature of not less than 37° C. (e.g., 42° C.), it is dissociated from the operator to allow for induction of gene expression. Therefore, the period when the expression of the target gene is suppressed can be minimized by culturing a host cell introduced with a DNA encoding the complex of the present invention generally at not more than 30° C., raising the temperature to not less than 37° C. at an appropriate stage, performing culture for a given period to cause expression of the complex of the present invention and a DNA modification reaction by a cell-endogenous DNA modifying enzyme recruited by the complex and, after introduction of mutation into the target gene, rapidly lowering the temperature to not more than 30° C. Thus, even when an essential gene for the host cell is targeted, it can be efficiently edited while suppressing the side effects.

When temperature sensitive mutation is utilized, for example, a temperature sensitive mutant of a protein necessary for autonomous replication of a vector is mounted on a vector containing a DNA encoding the complex of the present invention. As a result, autonomous replication cannot occur rapidly after expression of the complex, and the vector naturally falls off along with the cell division. Examples of such temperature sensitive mutant protein include, but are not limited to, a temperature sensitive variant of Rep101 ori necessary for replication of pSC101 ori. At not more than 30° C. (e.g., 28° C.), Rep101 ori (ts) acts on pSC101 ori to enable autonomous replication of plasmid. At not less than 37° C. (e.g., 42° C.), pSC101 ori loses its function and plasmid cannot replicate autonomously. Therefore, a combined use with cI repressor (ts) of the above-mentioned Aphage simultaneously enables transient expression of the complex of the present invention, and removal of the plasmid.

When a higher eukaryotic cell such as animal cell, insect cell, plant cell or the like is a host cell, a DNA encoding the complex of the present invention is introduced into the host cell under the control of an induction promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), an induction substance is added to (or removed from) the medium at an appropriate time to induce expression of the complex, the cells are cultured for a certain period to cause a DNA modification reaction by cell-endogenous DNA modifying enzyme recruited to the complex, and the aforementioned induction substance is removed from the medium after the mutation is introduced into the target gene, whereby a transient expression of the complex of the present invention can be realized.

An induction promoter can be utilized in prokaryotic cells such as Escherichia coli and the like. Examples of such induction promoter include, but are not limited to, lac promoter (induced by IPTG), cspA promoter (induced by cold shock), araBAD promoter (induced by arabinose) and the like.

Alternatively, the above-mentioned induction promoter can also be utilized as a vector removal mechanism when a higher eukaryotic cell such as animal cell, insect cell, plant cell or the like is a host cell. That is, a vector is loaded with a replication origin that functions in the host cell and a nucleic acid encoding a protein necessary for the replication (e.g., SV40 ori and large T antigen, oriP and EBNA-1 and the like for animal cells) and the expression of the nucleic acid encoding the protein is controlled by the above-mentioned induction promoter. The vector is autonomously replicatable in the presence of the induction substance, but cannot replicate autonomously when the induction substance is removed, and the vector falls off spontaneously along with cell division (Tet-OFF vector cannot replicate autonomously when tetracycline or doxycycline is added).

According to the studies conducted by the present inventors, once the expression and activity of a cell-endogenous DNA modifying enzyme is sufficiently increased using a DNA modifying enzyme inducer, the target DNA can be modified in some cases without using a DNA modifying enzyme-binding module but using only a nucleic acid sequence-recognizing module. While not wishing to be bound by any theory, a possible mechanism is that a DNA modifying enzyme present in a sufficient amount more frequently contacts the distortion of double helix structure in the target site which is caused by binding of the nucleic acid sequence-recognizing module, acts on the target site and alters the target DNA.

Therefore, in another embodiment of the present invention, a method for altering a targeted site of a DNA in a cell, comprising a step of stimulating the cell with a factor inducing a DNA modifying enzyme endogenous to the cell, and bringing a nucleic acid sequence-recognizing module specifically binding to a target nucleotide sequence in a given double stranded DNA into contact with the double stranded DNA to convert one or more nucleotides in the targeted site to other one or more nucleotides or delete one or more nucleotides, or insert one or more nucleotides into said targeted site is provided.

The present invention is explained in the following by referring to Examples, which are not to be construed as limitative.

### Examples

1. Vector construction
1-1. Cas9, nCas9, nCas9-dVif, dVif-nCas9 or nCas9-PmCDA1 expression vector

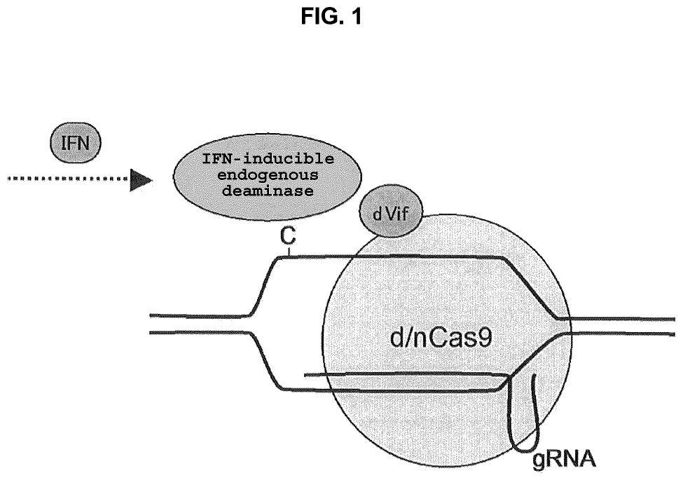

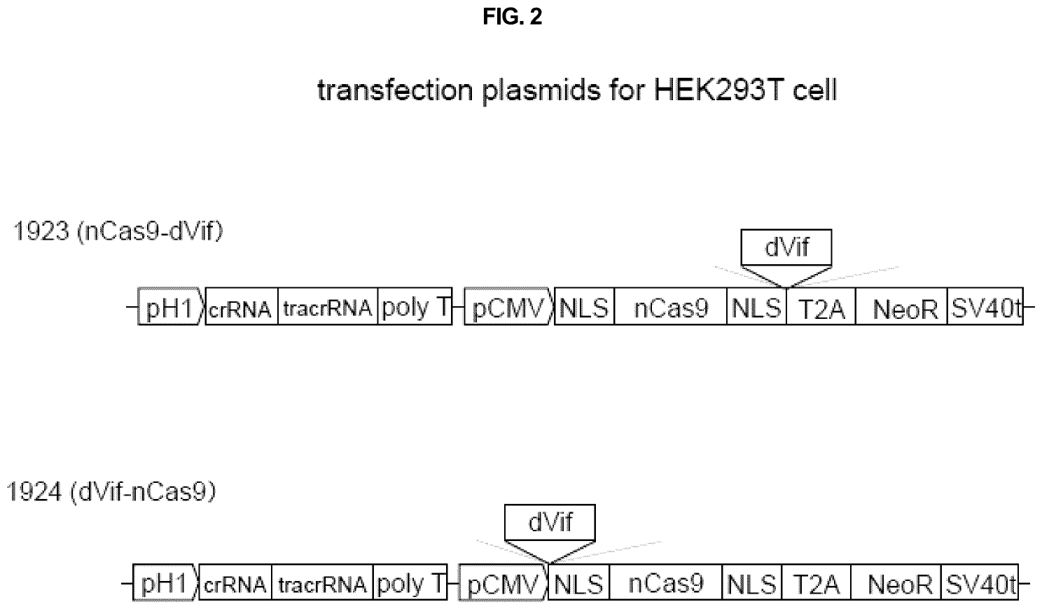

The outline of the plasmid vector for DNA editing used in the Examples is shown in FIG. 1. Using pNeo vector as a base, a plasmid vector for gene transgene was constructed by transfection into human fetal kidney-derived cells (HEK293T cells). As the plasmid vector, 1907c (Cas9), 1907n (nCas9-PmCDA1), 1907n-cugi (nCas9-PmCDA1-UGI), 1921 (nCas9), 1923 (nCas9-dVif), 1924 (dVif-nCas9) targeting Exon6 of hypoxanthine-guanine phosphoribosyl-transferase (HPRT) gene were used and pNeo was used as a control. 1907c (Cas9), 1907n (nCas9-PmCDA1), 1907n-cugi (nCas9-PmCDA1-UGI) and 1921 (nCas9) were constructed based on the vector used in non-patent document 3 and by changing the target sequence of guide RNA to the 24th—43rd sequence (aatgcagactttgctttcct: SEQ ID NO: 12) (site 3) from the start point of exon 6 of HPRT gene. As a DNA encoding nCas9 (D10A), a DNA consisting of the base sequence shown in SEQ ID NO: 4 was used. 1923 (nCas9-dVif) and 1924 (dVif-nCas9) were produced as follows. First, vector 1922 (SEQ ID NO: 13) was constructed by addition of a restriction enzyme site to and removal of unnecessary sequence from 1921 (nCas9). As for dVif fragment of HIV, reference was made to GenBank: AF200477.1 which is a Vif sequence on the database. In 28-576 bases of ORF of the aforementioned sequence, the 433rd—435th bases (CTA) were altered to GCT to synthesize an artificial gene introduced with L145A mutation (base sequence is shown in SEQ ID NO: 1, amino acid sequence is shown in SEQ ID NO: 2. A base sequence in which AvrII recognition site was added to 5′-side and NheI recognition site was added to 3′-side is shown in SEQ ID NO: 3), and the artificial gene was inserted in 1922 by cleavage of restriction enzyme and ligation to produce 1923 (nCas9-dVif) and 1924 (dVif-nCas9). FIG. 2 shows a schematic drawing of vectors 1923 (nCas9-dVif) and 1924 (dVif-nCas9) produced.

The aforementioned vectors were introduced into HEK293T cells and expressed in the cells to form a complex of crRNA-tracrRNA, and Cas9, nCas9, nCas9-dVif, dVif-nCas9 or nCas9-PmCDA1.

1-2. UGI-nCas9-dVif, dVif-nCas9-UGI, TopBv2(TopoIIβisoform 2)-nCas9, nCas9—IQGAP2₄₆₆₋₅₄₇—ZNF3₃₅₇₄₅₋₈₉₃ or nCas9-PmCDA1-UGI expression vector

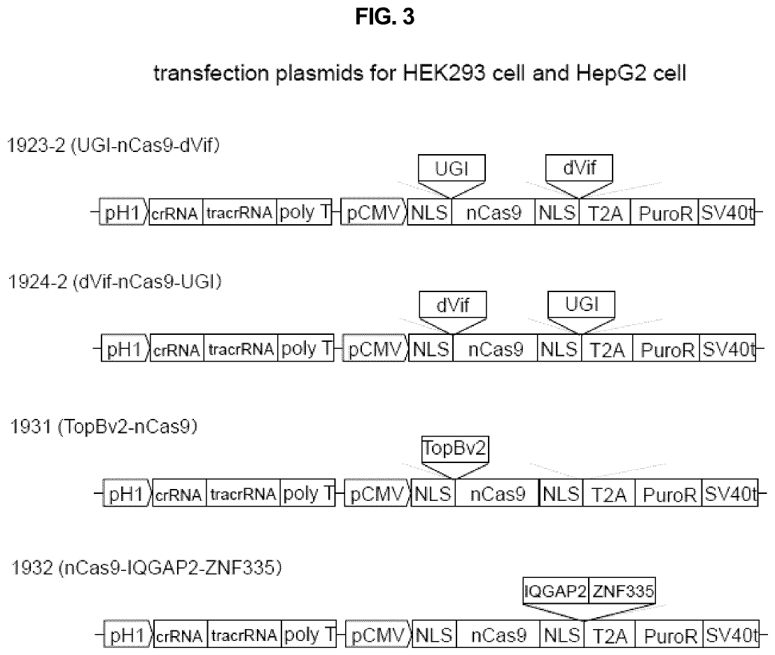

Referring to the procedure of 1-1., vector 1923-2 (UGI-nCas9-dVif: SEQ ID NO: 28), vector 1924-2 (dVif-nCas9-UGI: SEQ ID NO: 29), vector 1931 (TopBv2₄₅₂₋₅₉₁—nCas9: SEQ ID NO: 30) and vector 1932 (nCas9-IQGAP24₆₆₋₅₄₇-ZNF335₇₄₅₋₈₉₃: SEQ ID NO: 31) were produced each of which targets a particular region of HPRT gene (target sequence (site 1): tcgagatgtgatgaaggaga; SEQ ID NO: 27). In addition, vector 1907 (nCas9-PmCDA1-UGI: SEQ ID NO: 32) was produced for comparative testing. The base sequences encoding the fragments of TopBv2, IQGAP2 and ZNF335 were designed by reference to refseq No: NM 001068, NM 006633 and NM 022095, each of which is a sequence on the database. FIG. 3 shows a schematic drawing of vectors 1923-2, 1924-2, 1931 and 1932 produced. The base sequence encoding UGI and the amino acid sequence of UGI are respectively shown in SEQ ID NO: 19 and 20, the base sequence encoding TopBv2₄₅₂₋₅₉₁ and the amino acid sequence of TopBv2₄₅₂₋₅₉₁ are respectively shown in SEQ ID NO: 21 and 22, the base sequence encoding IQGAP2₄₆₆₋₅₄₇ and the amino acid sequence of IQGAP2₄₆₆₋₅₄₇ are respectively shown in SEQ ID NO: 23 and 24, and the base sequence encoding ZNF335₇₄₅₋₈₉₃ and the amino acid sequence of ZNF335₇₄₅₋₈₉₃ are respectively shown in SEQ ID NO: 25 and 26.

2. Cell line, culture, transformation, expression induction
2-1. Introduction system of vector of 1-1

The experiment using the vector of the above-mentioned 1-1 was performed by the following procedure. Human fetal kidney-derived cells (HEK293T cells) were used. The cells were cultured in a DME-glutamax medium (Thermo Fisher Scientific, USA) added with 100 μg/mL penicillin-streptomycin (Life Technologies, Carlsbad, CA, USA) and 10% fetal bovine serum (FBS) (Biosera, Nuaille, France) under 37° C., 5% CO₂ conditions. The cells were recovered using 5% trypsin.

HEK293T cells preserved in a deep freezer were dissolved in a water bath at 37° C. and seeded in a 75 T-flask at 5×10⁶ cells. After culturing for 1-3 days, the cells were recovered and seeded in each well of a 24 well plate at 0.5×10⁵ cells/well. After culturing for 1-3 days, about 1 μg of each of the above-mentioned plasmid DNAs was transfected into 60-80% confluent cells in each well by using 3 μl of Lipofectamine 2000 (Life Technologies, Carlsbad, USA). After 5 hours of transfection, the medium was replaced with one containing G418 (0.125 mg/mL) (InvivoGen, USA) and interferon α (IFNα) (2000 IU) (Takara Bio) or interferon γ (2000 IU) (PeproTech, Inc.). As a control, a medium containing G418 alone was used.

2-2. Introduction system of vector of 1-2

The experiment using the vector of the above-mentioned 1-2 was performed by the following procedure. The cells (HEK293 or HepG2) were seeded in each well of a 24 well plate at 1×10⁵ cells/well and cultured overnight. Then, transfection (DNA 1 μg/well, FugeneHD 1.5 μl/well) was performed using FugeneHD (Promega) and the medium was replaced 16 hr later. In the case of HEK293, OPTI-MEM was replaced with DMEM+10% FBS+P/S (penicillin-streptomycin)+Puromycin (1 μg/ml)+/− IFNα (10000 U/ml). In the case of HepG2, OPTI-MEM was replaced with DMEM+10% FBS+P/S+1% NEAA (non-essential amino acid)+Puromycin (1 μg/ml)+/−IFNα (10000U/ml). Selection by puromycin was continued for 6 days. In this case, the medium was replaced every 48 hr.

3. Sequence analysis
3-1. Introduction system of vector of 1-1

Genomic DNA was extracted by the following procedure from the cells recovered in the above-mentioned 2-1 and the sequence was analyzed. For sequence analysis, each cell was recovered 3 days after culture and genomic DNA was extracted. Using the extracted genomic DNA as a template and forward primer (5′-ATTCCAGAATATCTCCATGTAGATTTTGGT-3′: SEQ ID NO: 14) and reverse primer (5′-AATTCCAGGAGGTCCAGATCTTCAGGGCCC-3′: SEQ ID NO: 15) targeting Exon 6 of HPRT gene, the target region was amplified. Using the amplified DNA fragment as a template and forward primer (5′-TOTTTCCCTACACGACGCTOTTCCGATCTATTCCAGAATATCTCCATGTAGATTTTGGT-3′:

SEQ ID NO: 16) and reverse primer (5′—GTGACTGGAGTTCAGACGTGTGCTOTTCCGATCTTTAGGCAAGGAAGTGACTGTAATTATGAG C-3′: SEQ ID NO: 17), an about 300 bp amplification fragment added with an adapter for NGS analysis was obtained. An index sequence was added to each sample and deep sequencing with paired-end was performed using MiSeq Reagent Kit v3 and MiSeq sequencing system (Illumina). CLC Genomics Workbench 7.0 (Filgen) was used for the analysis. The results are shown in Table 1. In the Table, indel shows insertion deletion and number shows a nucleotide substitution rate (%). When cells expressing a complex of nCas9 and dVif were cultured in the presence of interferon, insertion deletions and/or base substitution occurred, and the base substitution was mostly substitution from cytosine to thymine. The ratio of insertion deletion and base substitution was of the same level as that in the conventional method (Target-AID) using exogenous deaminase. Many substitutions of bases are observed in the 19th T and 20th C of the nucleic acid bases in the Table. These mutations are considered to be sequence errors because substitution is highly frequently seen also in pNeo.

TABLE 1

| | HPRT site3 | Indel (%) | A | C | A | A | T | G | C | A | G | A | C | T | T |
|---|---|---|---|---|---|---|---|---|---|---|---|---|---|---|---|
| control | Neo | 0 | | | | | | | | | | | | | |
| | Cas9 | 2.16 | | | | | | A 0.12 | T 0.12 | C 0.16 | C 0.2 | C 0.12 | A 0.15 | G 0.31 | A 0.73 |
| | | | | | | | | | | G 0.12 | T 0.81 | T 0.16 | | | C 0.49 |
| | | | | | | | | | | T 0.2 | | | | | G 0.49 |
| | nCas9 | 0 | | | | | | | | | | | | | |
| IFN | nCas9 with αIFN | 0 | | | | | | | | | | | | | |
| | nCas9 with γIFN | 0 | | | | | | | | | | | | | |
| dVif | nCas9-dVif | 0 | | | | | | | | | | | | | |
| IFN + | nCas9-dVif αIFN | 0 | | | | | | A 0.15 | | | | | | | |
| dVif | | | | | | | | T 0.15 | T 0.37 | | | | | | |
| | nCas9-dVif γIFN | 0.23 | | | | | | | | | | | | | |
| | | | | | | | | | T 0.22 | | | | | | |
| | dVif-nCas9 | 0 | | | | | | | | | | | | | |
| | dVif-nCas9 αIFN | 0.11 | | | | | | | | | | | | | |
| | | | | | | | | | T 0.33 | | | | | | |
| | dVif-nCas9 γIFN | 0 | | | | | | | | | | | | | |
| Target- | nCas9-PmCDA1 | 0.11 | | | | | | | | | | | | | |
| AID | | | | | | | | | T 0.36 | | | | | | |

| | HPRT site3 | Indel (%) | T | G | C | T | T | T | C | C | T | T | G | G | T |
|---|---|---|---|---|---|---|---|---|---|---|---|---|---|---|---|
| control | Neo | 0 | | | | | | A 0.66 | A 1.73 | | | | | | |
| | | | | | | | | C 0.33 | | | | | | | |
| | | | | | | | | G 0.8 | | | | | | | |
| | Cas9 | 2.16 | | A 0.12 | A 0.16 | | | A 0.64 | A 1.52 | | | | T 0.12 | T 0.1 | |
| | | | | | | | | C 0.28 | G 0.43 | | | | | | |
| | | | | | | | | G 0.65 | T 0.35 | | | | | | |
| | nCas9 | 0 | | | | | | A 0.74 | A 1.66 | | | | | | |
| | | | | | | | | C 0.32 | | | | | | | |
| | | | | | | | | G 0.69 | | | | | | | |
| IFN | nCas9 with αIFN | 0 | | | | | | A 0.71 | A 1.77 | | | | | | |
| | | | | | | | | C 0.28 | | | | | | | |
| | | | | | | | | G 0.76 | | | | | | | |
| | nCas9 with γIFN | 0 | | | | | | A 0.71 | A 1.71 | | | | | | |
| | | | | | | | | G 0.76 | | | | | | | |
| dVif | nCas9-dVif | 0 | | | | | | A 0.71 | A 1.64 | | | | | | |
| | | | | | | | | C 0.37 | | | | | | | |
| | | | | | | | | G 0.74 | | | | | | | |
| IFN + | nCas9-dVif αIFN | 0 | | | | | | A 0.74 | A 1.77 | | | | | | |
| dVif | | | | | | | | C 0.27 | | | | | | | |
| | | | | | | | | G 0.73 | | | | | | | |
| | nCas9-dVif γIFN | 0.23 | | | | | | A 0.63 | A 1.61 | | | | | | |
| | | | | | | | | C 0.34 | G 0.36 | | | | | | |
| | | | | | | | | G 0.74 | | | | | | | |
| | dVif-nCas9 | 0 | | | | | | A 0.65 | A1.72 | | | | | | |
| | | | | | | | | G 0.67 | | | | | | | |
| | dVif-nCas9 αIFN | 0.11 | | | | | | A 0.64 | A 1.7 | | | | | | |
| | | | | | | | | C 0.29 | T 0.38 | | | | | | |
| | | | | | | | | G 0.68 | | | | | | | |
| | dVif-nCas9 γIFN | 0 | | | | | | A 0.69 | A 1.72 | | | | | | |
| | | | | | | | | C 0.31 | G 0.42 | | | | | | |
| | | | | | | | | G 0.74 | | | | | | | |
| Target- | nCas9-PmCDA1 | 0.11 | | | | | | A 0.71 | A 1.67 | | | | | | |
| AID | | | | | | | | C 0.31 | G 0.41 | | | | | | |
| | | | | | | | | G 0.72 | | | | | | | |

3-2. Introduction system of vector of 1-2 Genomic DNA was extracted from the cells collected in the above-mentioned 2.2 and the sequence was analyzed. HEK293 cells were collected on day 6, HepG2 cells were collected after recovery culture for 48 hr, and genomic DNA was extracted using NucleoSpin Tissue XS (Takara Bio Inc.). 1st PCR (DNA polymerase: KOD FX NEO (Toyobo), primer set: forward primer (5′-TTTGGTACTTGTTCAGCTTTATTCAAGTGG-3′: SEQ ID NO: 33); reverse primer (5′-ACAATAGCTCTTCAGTCTGATAAAATCTAC-3′: SEQ ID NO: 34)) was performed, the band was confirmed by electrophoresis, and the PCR product was purified using Exo/Sap (Thermo Fisher Scientific) to give a 1100 bp amplification fragment. Then, using the PCR product after purification as a template, 2nd PCR (DNA polymerase: KOD FX NEO, primer set: forward primer (5′-TCTTTCCCTACACGACGCTCTTCCGATCT TAGGACTGAACGTCTTGCTC-3′: SEQ ID NO: 35); reverse primer (5′-GTGACTGGAGTTCAGACGTGTGCTCTTCCGATCT CAGTCATAGGAATGGATCTATCAC-3′: SEQ ID NO: 36)) was performed, the band was confirmed by electrophoresis, and the PCR product was purified using Exo/Sap to give a 220 bp amplification fragment. Furthermore, using the PCR product after purification as a template, 3rd PCR (Q5 DNA polymerase (New England Biolabs), primer set:SEQ ID NO: 14 and 15) was performed, and the PCR product was purified using AMPure XP (Beckman Coulter) to give an about 150 bp amplification fragment added with an adapter for NGS analysis. The band of the samples after purification using AMPure XP was confirmed by Multina (SHIMADZU Corporation). The samples were pooled by referring to the bands (concentrations) obtained by Multina and the concentration of the samples was measured using Qubit (Thermo Fisher Scientific). The samples were diluted to 10 nM and confirmed by Qubit to be at 10 nM. 10 nM samples were diluted to 1 nM and 1 nM samples were altered. Thereafter, the samples were diluted to 1.5 pM. 4 nM PhiX (Illumina) was altered and diluted to 1.5 pM. 500 μl of the sample (1.5 pM) and 100 μl of PhiX (1.5 pM) were mixed and applied to a cartridge. Miniseq (Illumina) was started to perform sequencing. The results are shown in Table 2. In the Table, indel shows insertion deletion (indel was not detected in Table 2) and number shows substitution rate (%) of nucleotide. When HEK293 cells made to express a complex of UGI, nCas9 and dVif were cultured in the presence of interferon, base substitution from cytosine to thymine occurred. Similarly, when cells made to express a complex of nCas9 and TopBv2 or IQGAP2 and ZNF335 were cultured in the presence of interferon, base substitution from cytosine to thymine occurred. In addition, when HepG2 cells made to express a complex of UGI, nCas9 and dVif were cultured in the presence of interferon, base substitution from cytosine to thymine occurred. When HepG2 cells were used, the rate of base substitution was of the same level as the conventional method using exogenous deaminase (Target-AID).

TABLE 2
Hek293 cell HPRT site1
1923-2: UGI-nCas9-Vif

| | | | | | | | | | | | | | | | | | | | | | | | |
|---|---|---|---|---|---|---|---|---|---|---|---|---|---|---|---|---|---|---|---|---|---|---|---|
| | | 21 | 20 | 19 | 18 | 17 | 16 | 15 | 14 | 13 | 12 | 11 | 10 | 9 | 8 | 7 | 6 | 5 | 4 | 3 | 2 | 1 | PAM |
| 24 | | C | T | C | G | A | G | A | T | G | T | G | A | T | G | A | A | G | G | A | G | A | TGG |
| 1923-2 | C > T | 0.99 | | | | | | | | | | | | | | | | | | | | | |
| IFNα | C > G | | | | | | | | | | | | | | | | | | | | | | |
| | Indel | | | | | | | | | | | | | | | | | | | | | | |

1924-2: Vif-nCas9-UGI

| | | | | | | | | | | | | | | | | | | | | | | | |
|---|---|---|---|---|---|---|---|---|---|---|---|---|---|---|---|---|---|---|---|---|---|---|---|
| | | 21 | 20 | 19 | 18 | 17 | 16 | 15 | 14 | 13 | 12 | 11 | 10 | 9 | 8 | 7 | 6 | 5 | 4 | 3 | 2 | 1 | PAM |
| 18 | | C | T | C | G | A | G | A | T | G | T | G | A | T | G | A | A | G | G | A | G | A | TGG |
| 1924-2 | C > T | 0.76 | | | | | | | | | | | | | | | | | | | | | |
| IFNα | C > G | | | | | | | | | | | | | | | | | | | | | | |
| | Indel | | | | | | | | | | | | | | | | | | | | | | |

1931: TopBv2-nCas9

| | | | | | | | | | | | | | | | | | | | | | | | |
|---|---|---|---|---|---|---|---|---|---|---|---|---|---|---|---|---|---|---|---|---|---|---|---|
| | | 21 | 20 | 19 | 18 | 17 | 16 | 15 | 14 | 13 | 12 | 11 | 10 | 9 | 8 | 7 | 6 | 5 | 4 | 3 | 2 | 1 | PAM |
| 31 | | C | T | C | G | A | G | A | T | G | T | G | A | T | G | A | A | G | G | A | G | A | TGG |
| 1931 | C > T | 0.8 | | | | | | | | | | | | | | | | | | | | | |
| IFNα | C > G | | | | | | | | | | | | | | | | | | | | | | |
| | Indel | | | | | | | | | | | | | | | | | | | | | | |

1932: nCas9-ZF

| | | | | | | | | | | | | | | | | | | | | | | | |
|---|---|---|---|---|---|---|---|---|---|---|---|---|---|---|---|---|---|---|---|---|---|---|---|
| | | 21 | 20 | 19 | 18 | 17 | 16 | 15 | 14 | 13 | 12 | 11 | 10 | 9 | 8 | 7 | 6 | 5 | 4 | 3 | 2 | 1 | PAM |
| 27 | | C | T | C | G | A | G | A | T | G | T | G | A | T | G | A | A | G | G | A | G | A | TGG |
| 1932 | C > T | 0.71 | | | | | | | | | | | | | | | | | | | | | |
| IFNα | C > G | | | | | | | | | | | | | | | | | | | | | | |
| | Indel | | | | | | | | | | | | | | | | | | | | | | |

HepG2 cell HPRT site1
1924-2: Vif-nCas9-UGI

| | | | | | | | | | | | | | | | | | | | | | | | |
|---|---|---|---|---|---|---|---|---|---|---|---|---|---|---|---|---|---|---|---|---|---|---|---|
| | | 21 | 20 | 19 | 18 | 17 | 16 | 15 | 14 | 13 | 12 | 11 | 10 | 9 | 8 | 7 | 6 | 5 | 4 | 3 | 2 | 1 | PAM |
| 40 | | C | T | C | G | A | G | A | T | G | T | G | A | T | G | A | A | G | G | A | G | A | TGG |
| 1924-2 | C > T | 2.22 | | | | | | | | | | | | | | | | | | | | | |
| IFNα | C > G | | | | | | | | | | | | | | | | | | | | | | |
| | Indel | | | | | | | | | | | | | | | | | | | | | | |

1907: nCas9-CDA-ugi (reference data)

| | | | | | | | | | | | | | | | | | | | | | | | |
|---|---|---|---|---|---|---|---|---|---|---|---|---|---|---|---|---|---|---|---|---|---|---|---|
| | | 21 | 20 | 19 | 18 | 17 | 16 | 15 | 14 | 13 | 12 | 11 | 10 | 9 | 8 | 7 | 6 | 5 | 4 | 3 | 2 | 1 | PAM |
| 37 | | C | T | C | G | A | G | A | T | G | T | G | A | T | G | A | A | G | G | A | G | A | TGG |
| 1907 | C > T | 2.14 | | 3.17 | | | | | | | | | | | | | | | | | | | |
| IFNα | C > G | | | 0.5 | | | | | | | | | | | | | | | | | | | |
| | Indel | | | | | | | | | | | | | | | | | | | | | | |

This application is based on a patent application No. 2017-056727 filed in Japan (filing date: Mar. 22, 2017), the contents of which are incorporated in full herein.

### [Industrial Applicability]

According to the present invention, DNA editing which is safe due to the non-use of an exogenous enzyme in an alteration reaction of DNA and improved in the delivery efficiency by miniaturization of a construct used for the DNA editing has become possible, and the present invention is extremely useful.

1. A complex comprising
a nucleic acid sequence-recognizing module specifically binding to a target nucleotide sequence in a DNA and
a DNA modifying enzyme-binding module bonded to each other,
wherein the nucleic acid sequence-recognizing module is a CRISPR-Cas system,
wherein at least one DNA cleavage ability of Cas is inactivated, and
wherein the complex converts one or more nucleotides in the targeted site to other one or more nucleotides or deletes one or more nucleotides, or inserts one or more nucleotides into the targeted site.

2. A nucleic acid encoding the complex according to claim 1.

3. The complex according to claim 1, wherein the nucleic acid sequence-recognizing module is a CRISPR-Cas system wherein at least one DNA cleavage ability of Cas is inactivated.

4. The complex according to claim 3, wherein the nucleic acid sequence-recognizing module comprises a Cas9 and a guide RNA.

5. The complex according to claim 1, wherein the DNA modifying enzyme-binding module is selected from the group consisting of an antibody against the DNA modifying enzyme, a peptide aptamer against the DNA modifying enzyme and a nucleic acid aptamer against the DNA modifying enzyme.

6. The complex according to claim 1, wherein the DNA modifying enzyme-binding module is at least one kind selected from the group consisting of Virion infectivity factor (Vif), Bet protein, Topoisomerase 2-beta (Topollβ), IQ motif-containing GTPase activating protein 2 (IQGAP2) and Zinc finger protein 335 (ZNF335) and fragments thereof that bind to the DNA modifying enzyme.

7. The complex according to claim 1, wherein the DNA modifying enzyme-binding module is deaminase.

8. The complex according to claim 7, wherein the deaminase a protein belonging to apolipoprotein B mRNA-editing enzyme catalytic (APOBEC) cytidine deaminase.

9. The complex according to claim 1, wherein the nucleic acid sequence-recognizing module bonded to the DNA modifying enzyme-binding module further comprises a base excision repair inhibitor bonded thereto.

10. The complex according to claim 1, wherein the targeted site is altered without cleaving at least one strand of the DNA.

11. The complex according to claim 1, wherein the complex comprises a fusion protein, wherein the fusion protein comprises the nucleic acid sequence-recognizing module and the DNA modifying enzyme-binding module.


SEQUENCE LISTING (37 sequences)

SEQ ID NO: 1 - DNA, 549 bp, Human immunodeficiency virus 1
  misc_difference 406..408: note = artificial mutation
  source 1..549: mol_type = other DNA
  CDS 1..549
gtgtggcaagtagacaggatgaggattagaacatggaacagtttagtaaaacatcacatgtatatctcaaagaaagcaaaaaattggttttatagacatcactttgaaagcagtcatccaagagtaagttcagaagtacacatcccactaggggatgctagattagtagtaagaacatattggggtctgcatacaggagaaaaagattggcacttgggtaatggggtgtccatagaatggagactaagaagatatagcacacaaatagatcctgacctggcagaccaactaattcatctgcattattttaattgtttttcagactctgccataaggaaagccatattaggacaagtagttagacctagatgtgactatcaagcaggacataacaaggtaggatctgctcaatatttggcactgaaagcattagtaacaccagtaaggacaaggccacctttgcctagtgttaggaaattagcagaggacagatggaacaagccccagaaaaccaggggtcccagagggagccatacaatgaatggacat

SEQ ID NO: 2 - protein, 183 aa, Human immunodeficiency virus 1
VWQVDRMRIRTWNSLVKHHMYISKKAKNWFYRHHFESSHPRVSSEVHIPLGDARLVVRTYWGLHTGEKDWHLGNGVSIEWRLRRYSTQIDPDLADQLIHLHYFNCFSDSAIRKAILGQVVRPRCDYQAGHNKVGSAQYLALKALVTPVRTRPPLPSVRKLAEDRWNKPQKTRGPRGSHTMNGH

SEQ ID NO: 3 - DNA, 573 bp, synthetic construct
  misc_feature 1..573: note = Synthetic Sequence Vif variant construct
  source 1..573: mol_type = other DNA
cctaggggtaccgaaatggtgtggcaagtagacaggatgaggattagaacatggaacagtttagtaaaacatcacatgtatatctcaaagaaagcaaaaaattggttttatagacatcactttgaaagcagtcatccaagagtaagttcagaagtacacatcccactaggggatgctagattagtagtaagaacatattggggtctgcatacaggagaaaaagattggcacttgggtaatggggtgtccatagaatggagactaagaagatatagcacacaaatagatcctgacctggcagaccaactaattcatctgcattattttaattgtttttcagactctgccataaggaaagccatattaggacaagtagttagacctagatgtgactatcaagcaggacataacaaggtaggatctgctcaatatttggcactgaaagcattagtaacaccagtaaggacaaggccacctttgcctagtgttaggaaattagcagaggacagatggaacaagccccagaaaaccaggggtcccagagggagccatacaatgaatggacatgctagc

SEQ ID NO: 4 - DNA, 4116 bp, synthetic construct
  misc_feature 1..4116: note = Synthetic Sequence Cas9 codon-optimized for human.
  source 1..4116: mol_type = other DNA
  CDS 1..4116
atggacaagaagtactccattgggctcgmtatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatsmtatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgac

SEQ ID NO: 5 - protein, 1372 aa, synthetic construct
  SITE 10: note = misc_feature - The 'Xaa' at location 10 stands for Asp, or Ala.
  SITE 840: note = misc_feature - The 'Xaa' at location 840 stands for Asp, Ala, His, or Pro.
  REGION 1..1372: note = Synthetic Construct
MDKKYSIGLXIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDXIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD

SEQ ID NO: 6 - DNA, 21 bp, synthetic construct
  misc_feature 1..21: note = Synthetic Sequence SV40-derived nuclear localization signal
  source 1..21: mol_type = other DNA
  CDS 1..21
cctaagaagaarmgkaargtw

SEQ ID NO: 7 - protein, 7 aa, synthetic construct
  SITE 5: note = misc_feature - The 'Xaa' at location 5 stands for Arg, or Ser.
  SITE 7: note = misc_feature - The 'Xaa' at location 7 stands for Val.
  REGION 1..7: note = Synthetic Construct
PKKKXKX

SEQ ID NO: 8 - DNA, 51 bp, synthetic construct
  misc_feature 1..51: note = Synthetic Sequence SV40-derived nuclear localization signal
  misc_feature 6: note = n is a, c, g, or t
  misc_feature 9: note = n is a, c, g, or t
  misc_feature 27: note = n is a, c, g, or t
  misc_feature 30: note = n is a, c, g, or t
  misc_feature 39: note = n is a, c, g, or t
  misc_feature 42: note = n is a, c, g, or t
  misc_feature 45: note = n is a, c, g, or t
  misc_feature 48: note = n is a, c, g, or t
  misc_feature 51: note = n is a, c, g, or t
  source 1..51: mol_type = other DNA
  CDS 1..51
atggcnccnaaraaraaragraargtnggnathcayggngtnccngcngcn

SEQ ID NO: 9 - protein, 17 aa, synthetic construct
  REGION 1..17: note = Synthetic Construct
MAPKKKRKVGIHGVPAA

SEQ ID NO: 10 - RNA, 19 nt, Francisella tularensis
  misc_structure 1..19: note = crRNA direct repeat sequence.
  source 1..19: mol_type = other RNA; sub_species = novicida
aatttctactgttgtagat

SEQ ID NO: 11 - DNA, 76 bp, Streptococcus pyogenes
  misc_structure 1..76: note = tracrRNA
  source 1..76: mol_type = other DNA
gttttagagctagaaatagcaagttaaaataaggctagtccgttatcaacttgaaaaagtggcaccgagtcggtgc

SEQ ID NO: 12 - DNA, 20 bp, Homo sapiens
  source 1..20: mol_type = other DNA
aatgcagactttgctttcct

SEQ ID NO: 13 - DNA, 8604 bp, synthetic construct
  misc_feature 1..8604: note = Synthetic Sequence Complete nucleotide sequence of vector 1922
  regulatory 2233..2461: note = promoter - H1 promoter; regulatory_class = promoter
  misc_feature 2462..2481: note = Target(HPRT site 3)
  misc_structure 2482..2557: note = tracrRNA
  regulatory 2572..2826: note = promoter - CMV promoter; regulatory_class = promoter
  misc_feature 3232..3237: note = NheI recognition site
  misc_feature 3241..3291: note = NLS
  gene 3292..7407: note = nCas9
  misc_feature 7408..7428: note = NLS
  misc_feature 7444..7449: note = AvrII recognition site
  misc_feature 7453..7506: note = T2A
  gene 7513..7767: note = NeoR
  source 1..8604: mol_type = other DNA
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccggtgtaccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgaggaccacagatccccaatgcagactttgctttcctgttttagagctagaaatagcaagttaaaataaggctagtccgttatcaacttgaaaaagtggcaccgagtcggtgctttttttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgaaatggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggtacccctaggaccgaaggcaggggaagccttctgacttgtggggatgtggaagaaaaccctggtccatctagaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttagc

SEQ ID NO: 14 - DNA, 30 bp, synthetic construct
  misc_feature 1..30: note = Primer
  source 1..30: mol_type = other DNA
attccagaatatctccatgtagattttggt

SEQ ID NO: 15 - DNA, 30 bp, synthetic construct
  misc_feature 1..30: note = Primer
  source 1..30: mol_type = other DNA
aattccaggaggtccagatcttcagggccc

SEQ ID NO: 16 - DNA, 59 bp, synthetic construct
  misc_feature 1..59: note = Primer
  source 1..59: mol_type = other DNA
tctttccctacacgacgctcttccgatctattccagaatatctccatgtagattttggt

SEQ ID NO: 17 - DNA, 64 bp, synthetic construct
  misc_feature 1..64: note = Primer
  source 1..64: mol_type = other DNA
gtgactggagttcagacgtgtgctcttccgatctttaggcaaggaagtgactgtaattatgagc

SEQ ID NO: 18 - DNA, 26 bp, Homo sapiens
  source 1..26: mol_type = other DNA
acaatgcagactttgctttccttggt

SEQ ID NO: 19 - DNA, 252 bp, synthetic construct
  misc_feature 1..252: note = Synthetic Sequence PBS2-derived Ugi CDS optimized for eucaryotic cell expression
  source 1..252: mol_type = other DNA
  CDS 1..252
atgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgcta

SEQ ID NO: 20 - protein, 84 aa, synthetic construct
  REGION 1..84: note = Synthetic Construct
MTNLSDIIEKETGKQLVIQESILMLPEEVEEVIGNKPESDILVHTAYDESTDENVMLLTSDAPEYKPWALVIQDSNGENKIKML

SEQ ID NO: 21 - DNA, 420 bp, Homo sapiens
  source 1..420: mol_type = other DNA
aaaggtattcccaaactggatgatgctaatgatgctggtggtaaacattccctggagtgtacactgatattaacagagggagactctgccaaatcactggctgtgtctggattaggtgtgattggacgagacagatacggagtttttccactcaggggcaaaattcttaatgtacgggaagcttctcataaacagatcatggaaaatgctgaaataaataatattattaaaatagttggtctacaatataagaaaagttacgatgatgcagaatctctgaaaaccttacgctatggaaagattatgattatgaccgatcaggatcaagatggttctcacataaaaggcctgcttattaatttcatccatcacaattggccatcacttttgaagcatggttttcttgaagagttcattact

SEQ ID NO: 22 - protein, 140 aa, Homo sapiens
KGIPKLDDANDAGGKHSLECTLILTEGDSAKSLAVSGLGVIGRDRYGVFPLRGKILNVREASHKQIMENAEINNIIKIVGLQYKKSYDDAESLKTLRYGKIMIMTDQDQDGSHIKGLLINFIHHNWPSLLKHGFLEEFIT

SEQ ID NO: 23 - DNA, 246 bp, Homo sapiens
  source 1..246: mol_type = other DNA
gaagggaatcctttgaggactttagaaactttgctcctacctactgcgaatattagtgatgtggacccagcccatgcccagcactaccaggatgttttataccatgctaaatcacagaaactcggagactctgagagtgtttccaaagtgctttggctggatgagatacagcaagccgtcgatgatgccaacgtggacaaggacagagcaaaacaatgggttactctggtggttgatgttaatcag

SEQ ID NO: 24 - protein, 82 aa, Homo sapiens
EGNPLRTLETLLLPTANISDVDPAHAQHYQDVLYHAKSQKLGDSESVSKVLWLDEIQQAVDDANVDKDRAKQWVTLVVDVNQ

SEQ ID NO: 25 - DNA, 447 bp, Homo sapiens
  source 1..447: mol_type = other DNA
agttccccaggacctcctgagatacccccagaggcgacaactttccagtcatctgaggctccctcattgctctgttctgacaccctgggcggcgccaccatcatctaccagcaaggagctgaggagtcgacagcgatggccacgcagacagccttggatcttctgctgaacatgagtgctcagcgggaactggggggcacagccctgcaggtggctgtggtgaagtcggaagatgtggaagcagggttagcatcccctggtgggcagccctcccctgaaggtgccactccacaggtcgtcaccctccacgtggcagagccagggggcggtgcagcagccgagagccagctaggccctcctgacctaccgcagatcaccctggcacctggtccatttggtgggactggctacagtgtcatcacagcaccccctatggaggagggaaca

SEQ ID NO: 26 - protein, 149 aa, Homo sapiens
SSPGPPEIPPEATTFQSSEAPSLLCSDTLGGATIIYQQGAEESTAMATQTALDLLLNMSAQRELGGTALQVAVVKSEDVEAGLASPGGQPSPEGATPQVVTLHVAEPGGGAAAESQLGPPDLPQITLAPGPFGGTGYSVITAPPMEEGT

SEQ ID NO: 27 - DNA, 20 bp, Homo sapiens
  source 1..20: mol_type = other DNA
tcgagatgtgatgaaggaga

SEQ ID NO: 28 - DNA, 9228 bp, synthetic construct
  misc_feature 1..9228: note = Synthetic Sequence Complete nucleotide sequence of vector 1923-2
  misc_feature 167..188: note = CAP binding site
  rep_origin 476..1064: note = ori
  gene 1235..2095: note = AmpR
  regulatory 2096..2200: note = promoter - AmpR promoter; regulatory_class = promoter
  regulatory 2233..2461: note = promoter - H1 promoter; regulatory_class = promoter
  misc_feature 2462..2481: note = Target (HPRT site 1)
  misc_structure 2482..2557: note = tracrRNA
  regulatory 2572..2951: note = enhancer - CMV enhancer; regulatory_class = enhancer
  regulatory 2952..3155: note = promoter - CMV promoter; regulatory_class = promoter
  regulatory 3200..3218: note = promoter - T7 promoter; regulatory_class = promoter
  gene 3247..3498: note = UGI
  misc_feature 3502..3504: note = kozac
  misc_feature 3505..3555: note = NLS unit
  misc_feature 3511..3531: note = SV40 NLS
  gene 3556..7692: note = nCas9 (D10A)
  misc_feature 7672..7692: note = SV40 NLS
  gene 7714..8262: note = dVif
  misc_feature 7714..7722: note = CBF beta binding
  misc_feature 8272..8325: note = T2A
  gene 8332..8931: note = PuroR
  regulatory 9105..9226: note = polyA_signal - SV40 poly(A) signal; regulatory_class = polyA_signal_sequence
  source 1..9228: mol_type = other DNA
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccggtgtaccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgaggaccacagatcccctcgagatgtgatgaaggagagttttagagctagaaatagcaagttaaaataaggctagtccgttatcaacttgaaaaagtggcaccgagtcggtgctttttttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctaggggtaccgaaatgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctagccgaaatggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggtaccgaaatggtgtggcaagtagacaggatgaggattagaacatggaacagtttagtaaaacatcacatgtatatctcaaagaaagcaaaaaattggttttatagacatcactttgaaagcagtcatccaagagtaagttcagaagtacacatcccactaggggatgctagattagtagtaagaacatattggggtctgcatacaggagaaaaagattggcacttgggtaatggggtgtccatagaatggagactaagaagatatagcacacaaatagatcctgacctggcagaccaactaattcatctgcattattttaattgtttttcagactctgccataaggaaagccatattaggacaagtagttagacctagatgtgactatcaagcaggacataacaaggtaggatctgctcaatatttggcactgaaagcattagtaacaccagtaaggacaaggccacctttgcctagtgttaggaaattagcagaggacagatggaacaagccccagaaaaccaggggtcccagagggagccatacaatgaatggacatgctaggaccgaaggcaggggaagccttctgacttgtggggatgtggaagaaaaccctggtccatctagaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttagc

SEQ ID NO: 29 - DNA, 9198 bp, synthetic construct
  misc_feature 1..9198: note = Synthetic Sequence Complete nucleotide sequence of vector 1924-2
  rep_origin 476..1064: note = ori
  gene 1235..2095: note = AmpR
  regulatory 2096..2200: note = promoter - AmpR promoter; regulatory_class = promoter
  regulatory 2233..2461: note = promoter - H1 promoter; regulatory_class = promoter
  gene 2462..2481: note = HPRT exon
  misc_feature 2462..2481: note = Target (HPRT site 1)
  misc_structure 2482..2557: note = tracrRNA
  regulatory 2572..2951: note = enhancer - CMV enhancer; regulatory_class = enhancer
  regulatory 2952..3155: note = promoter - CMV promoter; regulatory_class = promoter
  regulatory 3200..3218: note = promoter - T7 promoter; regulatory_class = promoter
  gene 3250..3798: note = dVif
  misc_feature 3250..3258: note = CBF beta binding
  misc_feature 3805..3807: note = kozac
  misc_feature 3808..3858: note = NLS unit
  misc_feature 3814..3834: note = SV40 NLS
  gene 3859..7962: note = nCas9 (D10A)
  misc_feature 7963..7983: note = SV40 NLS
  gene 7990..8241: note = UGI
  misc_feature 8242..8295: note = T2A
  gene 8302..8901: note = PuroR
  regulatory 9075..9196: note = polyA_signal - SV40 poly(A) signal; regulatory_class = polyA_signal_sequence
  source 1..9198: mol_type = other DNA
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccggtgtaccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgaggaccacagatcccctcgagatgtgatgaaggagagttttagagctagaaatagcaagttaaaataaggctagtccgttatcaacttgaaaaagtggcaccgagtcggtgctttttttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctaggggtaccgaaatggtgtggcaagtagacaggatgaggattagaacatggaacagtttagtaaaacatcacatgtatatctcaaagaaagcaaaaaattggttttatagacatcactttgaaagcagtcatccaagagtaagttcagaagtacacatcccactaggggatgctagattagtagtaagaacatattggggtctgcatacaggagaaaaagattggcacttgggtaatggggtgtccatagaatggagactaagaagatatagcacacaaatagatcctgacctggcagaccaactaattcatctgcattattttaattgtttttcagactctgccataaggaaagccatattaggacaagtagttagacctagatgtgactatcaagcaggacataacaaggtaggatctgctcaatatttggcactgaaagcattagtaacaccagtaaggacaaggccacctttgcctagtgttaggaaattagcagaggacagatggaacaagccccagaaaaccaggggtcccagagggagccatacaatgaatggacatgctagcgaaatggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagaccccaagaagaaaagaaaagtcggtaccatgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctagaaggcaggggaagccttctgacttgtggggatgtggaagaaaaccctggtccatctagaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttagc

SEQ ID NO: 30 - DNA, 8859 bp, synthetic construct
  misc_feature 1..8859: note = Synthetic Sequence Complete nucleotide sequence of vector 1931
  rep_origin 476..1064: note = ori
  gene 1235..2095: note = AmpR
  regulatory 2096..2200: note = promoter - AmpR promoter; regulatory_class = promoter
  regulatory 2233..2461: note = promoter - H1 promoter; regulatory_class = promoter
  misc_feature 2462..2481: note = Target (HPRT site 1)
  misc_structure 2482..2557: note = tracrRNA
  regulatory 2572..2951: note = enhancer - CMV enhancer; regulatory_class = enhancer
  regulatory 2952..3155: note = promoter - CMV promoter; regulatory_class = promoter
  gene 3253..3672: note = fragment of TopBv2 (452-591)
  misc_feature 3691..3741: note = NLS unit
  misc_feature 3697..3717: note = SV40 NLS
  gene 3742..7878: note = nCas9 (D10A)
  misc_feature 7858..7878: note = SV40 NLS
  misc_feature 7903..7956: note = T2A
  gene 7963..8562: note = PuroR
  regulatory 8736..8857: note = polyA_signal - SV40 poly(A) siglan; regulatory_class = polyA_signal_sequence
  source 1..8859: mol_type = other DNA
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccggtgtaccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgaggaccacagatcccctcgagatgtgatgaaggagagttttagagctagaaatagcaagttaaaataaggctagtccgttatcaacttgaaaaagtggcaccgagtcggtgctttttttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgaaatgggtaccggaaaaggtattcccaaactggatgatgctaatgatgctggtggtaaacattccctggagtgtacactgatattaacagagggagactctgccaaatcactggctgtgtctggattaggtgtgattggacgagacagatacggagtttttccactcaggggcaaaattcttaatgtacgggaagcttctcataaacagatcatggaaaatgctgaaataaataatattattaaaatagttggtctacaatataagaaaagttacgatgatgcagaatctctgaaaaccttacgctatggaaagattatgattatgaccgatcaggatcaagatggttctcacataaaaggcctgcttattaatttcatccatcacaattggccatcacttttgaagcatggttttcttgaagagttcattactggaggtgaccctagcgaaatggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggtacccctaggaccgaaggcaggggaagccttctgacttgtggggatgtggaagaaaaccctggtccatctagaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttagc

SEQ ID NO: 31 - DNA, 9141 bp, synthetic construct
  misc_feature 1..9141: note = Synthetic Sequence Complete nucleotide sequence of vector 1932
  rep_origin 476..1064: note = ori
  gene 1235..2095: note = AmpR
  regulatory 2096..2200: note = promoter - AmpR promoter; regulatory_class = promoter
  regulatory 2233..2461: note = promoter - H1 promoter; regulatory_class = promoter
  misc_feature 2462..2481: note = Target (HPRT site 1)
  misc_structure 2482..2557: note = tracrRNA
  regulatory 2572..2951: note = enhancer - CMV enhancer; regulatory_class = enhancer
  regulatory 2952..3155: note = promoter - CMV promoter; regulatory_class = promoter
  misc_feature 3241..3291: note = NLS unit
  misc_feature 3247..3267: note = SV40 NLS
  gene 3292..7428: note = nCas9 (D10A)
  misc_feature 7408..7428: note = SV40 NLS
  gene 7447..7692: note = fragment of IQGAP2 (466-547)
  gene 7717..8163: note = fragment of NIF-ZFN335_745-893 (466-547)
  misc_feature 8185..8238: note = T2A
  gene 8245..8844: note = PuroR
  regulatory 9018..9139: note = polyA_signal - SV40 poly(A) signal; regulatory_class = polyA_signal_sequence
  source 1..9141: mol_type = other DNA
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccggtgtaccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgaggaccacagatcccctcgagatgtgatgaaggagagttttagagctagaaatagcaagttaaaataaggctagtccgttatcaacttgaaaaagtggcaccgagtcggtgctttttttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgaaatggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggtaccggagaagggaatcctttgaggactttagaaactttgctcctacctactgcgaatattagtgatgtggacccagcccatgcccagcactaccaggatgttttataccatgctaaatcacagaaactcggagactctgagagtgtttccaaagtgctttggctggatgagatacagcaagccgtcgatgatgccaacgtggacaaggacagagcaaaacaatgggttactctggtggttgatgttaatcagggaggccctagggaaatgggaggcagttccccaggacctcctgagatacccccagaggcgacaactttccagtcatctgaggctccctcattgctctgttctgacaccctgggcggcgccaccatcatctaccagcaaggagctgaggagtcgacagcgatggccacgcagacagccttggatcttctgctgaacatgagtgctcagcgggaactggggggcacagccctgcaggtggctgtggtgaagtcggaagatgtggaagcagggttagcatcccctggtgggcagccctcccctgaaggtgccactccacaggtcgtcaccctccacgtggcagagccagggggcggtgcagcagccgagagccagctaggccctcctgacctaccgcagatcaccctggcacctggtccatttggtgggactggctacagtgtcatcacagcaccccctatggaggagggaacaggagggtcacctactaggaccgaaggcaggggaagccttctgacttgtggggatgtggaagaaaaccctggtccatctagaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttagc

SEQ ID NO: 32 - DNA, 10219 bp, synthetic construct
  misc_feature 1..10219: note = Synthetic Sequence Complete nucleotide sequence of vector 1907
  gene 1238..2095: note = AmpR
  regulatory 2882..3110: note = promoter - H1 promoter; regulatory_class = promoter
  misc_feature 3111..3130: note = Target (HPRT site 1)
  misc_structure 3131..3206: note = tracrRNA
  regulatory 3221..3600: note = enhancer - CMV enhancer; regulatory_class = enhancer
  regulatory 3601..3804: note = promoter - CMV promoter; regulatory_class = promoter
  misc_feature 3890..3940: note = NLS unit
  misc_feature 3896..3916: note = SV40 NLS
  gene 3941..8077: note = nCas9 (D10A)
  misc_feature 8057..8077: note = SV40 NLS
  misc_feature 8108..8278: note = dead SH3 hs
  misc_feature 8285..8350: note = 3xFLAG hs
  gene 8357..8980: note = HsPmCDA1
  misc_feature 8984..9004: note = SV40 NLS
  gene 9011..9262: note = UGI
  misc_feature 9263..9316: note = T2A
  gene 9323..9922: note = PuroR
  regulatory 10096..10217: note = polyA_signal - SV40 poly(A) signal; regulatory_class = polyA_signal_sequence
  source 1..10219: mol_type = other DNA
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgaggaccacagatccccccgagatgtcatgaaagagagttttagagctagaaatagcaagttaaaataaggctagtccgttatcaacttgaaaaagtggcaccgagtcggtgctttttttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgaaatggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggtaccggcggtggaggctcagcagaatacgtacgagctctgtttgacttcaatgggaatgacgaggaggatctcccctttaagaagggcgatattctccgcatcagagataagcccgaagaacaatggtggaatgccgaggatagcgaagggaaaaggggcatgattctggtgccatatgtggagaaatattccggtgactacaaagaccatgatggggattacaaagaccacgacatcgactacaaagacgacgacgataaatcagggatgacagacgccgagtacgtgcgcattcatgagaaactggatatttacaccttcaagaagcagttcttcaacaacaagaaatctgtgtcacaccgctgctacgtgctgtttgagttgaagcgaaggggcgaaagaagggcttgcttttggggctatgccgtcaacaagccccaaagtggcaccgagagaggaatacacgctgagatattcagtatccgaaaggtggaagagtatcttcgggataatcctgggcagtttacgatcaactggtattccagctggagtccttgcgctgattgtgccgagaaaattctggaatggtataatcaggaacttcggggaaacgggcacacattgaaaatctgggcctgcaagctgtactacgagaagaatgcccggaaccagataggactctggaatctgagggacaatggtgtaggcctgaacgtgatggtttccgagcactatcagtgttgtcggaagattttcatccaaagctctcataaccagctcaatgaaaaccgctggttggagaaaacactgaaacgtgcggagaagcggagatccgagctgagcatcatgatccaggtcaagattctgcataccactaagtctccagccgttggtcccaagaagaaaagaaaagtcggtaccatgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctagaaggcaggggaagccttctgacttgtggggatgtggaagaaaaccctggtccatctagaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttagc

SEQ ID NO: 33 - DNA, 30 bp, synthetic construct
  misc_feature 1..30: note = Primer
  source 1..30: mol_type = other DNA
tttggtacttgttcagctttattcaagtgg

SEQ ID NO: 34 - DNA, 30 bp, synthetic construct
  misc_feature 1..30: note = Primer
  source 1..30: mol_type = other DNA
acaatagctcttcagtctgataaaatctac

SEQ ID NO: 35 - DNA, 49 bp, synthetic construct
  misc_feature 1..49: note = Primer
  source 1..49: mol_type = other DNA
tctttccctacacgacgctcttccgatcttaggactgaacgtcttgctc

SEQ ID NO: 36 - DNA, 58 bp, synthetic construct
  misc_feature 1..58: note = Primer
  source 1..58: mol_type = other DNA
gtgactggagttcagacgtgtgctcttccgatctcagtcataggaatggatctatcac

SEQ ID NO: 37 - DNA, 21 bp, Homo sapiens
  source 1..21: mol_type = other DNA
ctcgagatgtgatgaaggaga